(19) United States
(12) Patent Application Publication
(10) Pub. No.: US 2011/0196061 A1
(43) Pub. Date: Aug. 11, 2011

(54) INITIATORS AND CROSSLINKABLE POLYMERIC MATERIALS

(76) Inventors: Arthur Ashman, Westport, CT (US); V. Prasad Shastri, Nashville, TN (US)

(21) Appl. No.: 13/027,143

(22) Filed: Feb. 14, 2011

Related U.S. Application Data
(63) Continuation of application No. 11/240,747, filed on Sep. 30, 2005, now abandoned.
Application No. 11/240,747 is a continuation-in-part of application No. 10/789,442, filed on Feb. 26, 2004, now Pat. No. 7,393,493.
(60) Provisional application No. 60/450,538, filed on Feb. 27, 2003.

(51) Int. Cl. A61F 2/28

(52) U.S. Cl. 523/115

(57) ABSTRACT

The present invention relates to novel initiator systems, methods of use, and cured composition for dental, orthopedic and drug delivery purpose. Specifically, it relates to a crosslinkable prepolymer where crosslinking is initiated by a two part system and a composition comprising an admixture of a resorbable bone substitute and a crosslinkable prepolymer. It also relates to the composition formed by crosslinking the admixture and a delivery system for cross-linking the polymer.

This application claims priority to U.S. patent application Ser. No. 10/789,442 filed Feb. 26, 2004, herein incorporated by reference.

### FIELD OF THE INVENTION

The present invention relates generally to initiators, methods of use, and materials which may be used in any part of the body as an implant or graft material. Specifically, the invention relates to initiators for crosslinkable polymeric materials which can promote the formation of bone and/or other tissue(s) and the applications for such materials.

### BACKGROUND OF THE INVENTION

In the healing arts, there is often a need for an implant or graft material to replace, repair, or reconstruct tissues, in particular, hard tissues such as bone. For example, hard-tissue implant materials have been used in medicine and veterinary medicine as prosthetic bone materials to repair injured or diseased bone. Hard tissue implant materials are also used in the construction of prosthetic joints to fix the prosthetic joints to bones. In the dental art, hard tissue implant materials are used in the reconstruction of jaw bone damages caused by trauma, disease, or tooth loss; in the replacement or augmentation of the edentulous ridge; in the prevention of jaw bone loss by socket grafting; and in the treatment of periodontal bone void defects.

In orthopedics, hard tissue implant materials are used in the reconstruction of bone structure caused by trauma, disease, or surgery. For surgical procedures such as intervertebral diskectomy, the intervertebral disk is removed to provide access in removing the offending tissue, or bone osteophytes. In a spinal fusion procedure, it may be required to fix the vertebrae together to prevent movement and maintain a space originally occupied by the intervertebral disk.

During a spinal fusion following a diskectomy, a prosthetic implant or spinal implant is inserted into the intervertebral space. This prosthetic implant is often a bone graft material removed from another portion of the patient's body, termed an autograft. The use of bone taken from the patient's body has the important advantage of avoiding rejection of the implant, but has several shortcomings. There is always a risk in opening a second surgical site in obtaining the implant, which can lead to infection or pain for the patient, and the site of the implant is weakened by the removal of bony material. The bone implant may not be perfectly shaped and placed, leading to slippage or absorption of the implant, or failure of the implant to fuse with the vertebrae.

Other options for a graft source of the implant are bone removed from cadavers, termed allograft, or from other species, termed a xenograft. In these cases while there is the benefit of not having a second surgical site as a possible source of infection or pain, there is increased difficulty of the graft rejection and the risk of transmitting communicable diseases.

An alternative approach is using a bone graft or to use a manufactured implant made of a synthetic material that is biologically compatible with the body and the vertebrae. Over the last decade, polymeric materials have been used widely as bone graft materials. These materials are bio-inert, biocompatible, can serve as a temporary scaffold to be replaced by host tissue over time, and can be degraded by hydrolysis or by other means to non-toxic products.

Using these materials, various prosthetic implants can be generally divided into two basic categories, namely, solid implants and implants that are designed to encourage bone ingrowth. Implants that promote natural bone ingrowth achieve a more rapid and stable arthrodesis. Often, these implants are filled with autologous bone prior to insertion into the intervertebral disk space and include apertures which communicate with openings in the implant, thereby providing a path for tissue growth between the vertebral end plate and the bone or bone substitute within the implant. In preparing the intervertebral disk space for a prosthetic implant, the end plates of the vertebrae are preferably reduced to bleeding bone to facilitate tissue growth within the implant.

A number of difficulties still remain with the many prosthetic implants currently available. While it is recognized that hollow implants which permit bone ingrowth in the bone or bone substitute within the implant is an optimum technique for achieving fusion, most of these devices have difficulty achieving this fusion, at least without the aid of some additional stabilizing device, such as a rod or plate. Moreover, some of these devices are not structurally strong enough to support the heavy loads applied at the most frequently fused vertebral levels, mainly those in the lower lumbar spine.

In the dental art, when a tooth is extracted, a large cavity is created in the alveolar bone. The alveolar bone begins to undergo resorption at a rate of 40-60% in 2-3 years, which continues yearly at a rate of 0.25% to 0.50% per year until death (Ashman A. et al., Prevention of Alveolar Bone Loss Post Extraction with Bioplant® HTR® Grafting Material. Oral Surg. Oral. Med. Oral. Pathol. 60 (2):146-153, (1985)). Shifting of the remaining teeth, pocket formation, bulging out of the maxillary sinus, poor denture retention, loss of vertical dimension, formation of facial lines, unaesthetic gaps between bridgework and gum are some of the undesirable consequences associated with such loss (Luc. W. J. Huys, Hard Tissue Replacement, Dentist News, (Feb. 15, 2002)). Such bone loss also creates a significant problem for the placement of dental implants to replace the extracted tooth. It has been reported in previous years that nearly 95% of implant candidates rejected were turned down because of inadequate height and/or width of the alveolar bone (Ashman A., Ridge Preservation, Important Buzzwords in Dentistry, General Dentistry, May/June, (2000)).

One proven technique for overcoming the bone and soft tissue problems associated with the extraction of the tooth is to fill the extraction site with a bone graft material (e.g., synthetic, bovine or cadaver derived), and cover the site with gum tissue (e.g., suturing closed) or a dental “bandage” (e.g., Biofoil® Protective Stripes) for a period of time sufficient for new bone growth. The cavity becomes filled with a mixture of the bone graft material acting as an osteoconductive scaffold for the newly regenerated/generated bone. When implant placement is desired, after a period of time sufficient to allow bone regeneration (or healing) in the cavity, a cylindrical bore drill can prepare the former extraction site, and a dental implant can be installed in the usual manner.

The problem associated with such technique is that, with most bone graft materials (e.g., cadaver- and bovine-derived); the dental implant cannot be installed immediately and placed in function with a suitable crown after the tooth extraction. Patients need to have repeated visits to the dentist's office, often waiting up to 6 months before a functional crown can be placed. In recent years, it has been reported that, with a few bone graft materials such as the Bioplant® HTR® detailed below, an implant can be placed immediately post-extraction (Ashman A. et al., Ridge Augmentation For Immediately Postextraction Implants Eight-Year Retrospective Study, The Regeneration Report, 7 (2), 85-95, (1995); Yukna R. A. et al., Evaluation of Hard Tissue Replacement Composite Graft Material as a Ridge Preservation/Augmentation Material in Conjunction with Immediate Hydroxyapatite-Coated Dental Implants, J. Periodontol., pages 679-685, May 2003; and Yukna R. A. et al., Bioplant® HTR® Synthetic Bone Grafts and Immediate Dental Implants, Compendium of Continuing Education in Dentistry, pages 649-657, September 2003, 24 (9)). However, such immediate post-extraction implants were not immediately made functional with a crown to chew. A healing period of 4-8 months was typically required for bone generation around the implant before loading. In other words, for example, prior to the present invention, if a patient has to have a front tooth extracted and replaced, the best the dentist can do is to install a metal implant (e.g., titanium) immediately after the extraction, place a bone graft material (e.g., Bioplant® HTR® or a “barrier membrane”) around the implant in the socket and send him home. A crown cannot be installed on top of the metal implant until the implant becomes load-bearing (i.e., osteointegrated), months after the implant placement. In the meantime, the patient does not have a functional (e.g., cannot chew) or an esthetically-pleasing replacement tooth.

U.S. Pat. Nos. 4,535,485 and 4,536,158 disclose certain polymer-based implantable porous prostheses for use as bone or other hard tissue replacement which are composed generally of polymeric particles. Although the porous prostheses of the '485 and '158 patents have proven to be satisfactory for many applications in dentistry and orthopedics, there is room for improvement.

U.S. Pat. No. 4,728,570 discloses a porous implant material which induces the growth of hard tissue. Based on the '570 patent, Bioplant Inc. (South Norwalk, Conn.) manufactures a very slowly absorbable product called Bioplant® HTR® This product has proven to be very useful in both preventing bone loss and stimulating bone generation. It has also been found suitable for esthetic tissue plumping as well as for immediate post-extraction implants as mentioned above. However, it, like all bone graft materials prior to the present invention, when placed in an extraction socket or in edentulous spaces, the implant would not be immediately functional. A patient still must wait months for bone generation (e.g., osteointegration) to take place around the implant before revisiting the dentist's office months later to have a crown installed.

Within the last decade, polymers that are more biodegradable and/or bioresorbable than PMMA and PHEMA have been introduced into the field of tissue replacement.

Medical devices made with degradable polyesters such poly(L-lactic acid), poly(glycolic acid), and poly(lactic-co-glycolic acid) are approved for human use by the Food and Drug Administration, and have been used in many medical applications, for example, in sutures. These polymers, however, lack many properties necessary for restoring function in high load-bearing bone applications, since they undergo homogeneous, bulk degradation which is detrimental to the long-term mechanical properties of the material and leads to a large burst of acid products near the end of degradation (e.g., similar to inflammation). In contrast, surface eroding polymers (such as polyanhydrides) maintain their mechanical integrity during degradation and exhibit a gradual loss in size which permits bone ingrowth. However, linear polyanhydride systems have limited mechanical strength.

U.S. Pat. No. 5,837,752 discloses a semi-interpenetrating polymer network (“semi-IPN”) composition for bone repair comprising (1) a linear polymer selected from the group consisting of linear, hydrophobic biodegradable polymers and linear non-biodegradable hydrophilic polymers; and (2) one or more crosslinkable monomers or macromers containing at least one free radical polymerizable group, wherein at least one of the monomers or macromers includes an anhydride linkage and a polymerizable group selected from the group consisting of acrylate or methacrylate.

U.S. Pat. No. 5,902,599 discloses biodegradable polymer networks which are useful in a variety of dental and orthopedic applications. Such biodegradable polymer networks can be formed by polymerizing anhydride prepolymers containing crosslinkable groups, such as unsaturated moieties. The anhydride prepolymers can be crosslinked, for example in a photopolymerization reaction by irradiation of the prepolymer with light in the presence of a photosensitive free radical initiator.

WO 01/74411 discloses a composition suitable for preparing a biodegradable implant comprised of a crosslinkable multifunctional prepolymer having at least two polymerizable terminal groups. It discloses placing a metal screw implant immediately into the extraction socket; firmly packing the void between the bone and the implant with a graft material such as the Bioplant® HTR®; applying a layer of the crosslinkable multifunctional prepolymer on top of the graft material and curing the layer to form a rigid collar around the metal implant. The cured ring around the neck of the implant allegedly resists the chewing forces on the implant that are mainly concentrated at the neck of the implant. However, the alleged support and resistance provided by such a cured ring is not sufficient in either the short or the long term, since the implant is only secured around the neck which is a very narrow area near the gum line. Hence, even if the cured ring is hardened, it does not provide adequate rigidity in the short term. In the long term, the cured ring does not have sufficient bone regenerating capability due to the lack of a bone stimulation material. Hence, the implant is not stable, still exhibits significant micromovement, and is not immediately load-bearing. Accordingly, WO 01/74411 does not teach, suggest, or enable an immediately functional replacement tooth.

Therefore, there is a continued need in the replacement and restorative arts for materials and methods which reduce the time of the bone regenerative process, allow immediately functional dental implants, provide sufficient mechanical strength, and/or minimize micromovement. In addition, there is a need to broaden the spectra of materials available for dental and orthopedic implants and for bone substitutes that can be used for the delivery of therapeutic agents (i.e., bone growth factors).

### SUMMARY OF THE INVENTION

The present invention relates to novel methods, compositions, and processes for dental, orthopedic and drug delivery purposes. Specifically, it relates to novel initiator systems, methods of use, and curable and cured composition for dental, orthopedic and drug delivery purpose. Specifically, it relates to a crosslinkable prepolymer where crosslinking is initiated by a two part system.

Surprisingly, it has been discovered that the foregoing invention provides a curable admixture which immediately hardens upon curing and which becomes load-bearing so as to provide immediate support for, e.g., the installation of a crown and immediate functionality for the artificial tooth or for the spine after spinal fusion.

The initiator system comprises (i) an initiator component having a light radical generating component, a chemical radical generating component, and a solvent, (ii) an accelerator component comprising: a light accelerator component, a chemical accelerator component, and a solvent, wherein the initiator system is useful for initiating polymerization of a crosslinkable anhydride polymer system.

In one embodiment, the composition also comprises a bone substitute, which can be a ceramic, alloplast, autograft, allograft, xenograft, or a mixture thereof. Preferably, it is an alloplast; more preferably a polymeric alloplast (porous or non-porous); even more preferably porous micron-sized particles, wherein each particle comprises a core layer comprised of a first polymeric material and a coating generally surrounding the core layer, the coating comprising a second polymeric material, wherein the second polymeric material is hydrophilic and has a composition different from the composition of the first polymeric material, and both polymeric materials are biocompatible.

Preferably, the diameter of the micron-sized particles is in the range of from about 250 microns to about 900 microns.

Preferably, the first polymeric material is polymethylmethacrylate, the second polymeric material is a polymeric hydroxyethylmethacrylate; and the composition further comprises a quantity of calcium hydroxide distributed on the internal and external surfaces of the micron-sized particles of the bone substitute. Upon exposure to aqueous solution (e.g., blood), calcium hydroxide is converted to a calcium carbonate apatite (bone) compound.

The crosslinkable prepolymer comprises a monomer and/or oligomer having polymerizable group(s) to crosslink to form a polymer network.

There are three embodiments detailed for the crosslinkable prepolymer, with the first two being the most preferred. When cured, the hydrophobic nature of the polyanhydrides and the crosslinked structure keep water out of the interior of the polymer and allow for hydrolysis only at the surface. Hence, the polymer erodes only from the outside in. This type of degradation is particularly beneficial for dental, orthopedic and drug delivery applications because the cured composite will maintain structural integrity and/or mechanical integrity. In comparison, the polyorthoesters and polyacetals, etc., disclosed in the third embodiment below tend to degrade in a more homogeneous fashion because they are more hydrophilic, not as tightly crosslinked, and more susceptible to water penetration. The biodegradable bonds in the third embodiment, therefore, cleave internally as well as externally, leading to a more rapid loss in strength at the outset.

Optionally, the composition further comprises a therapeutic agent, a bone promoting agent, a porosity forming agent, or a diagnostic agent.

The curable admixture comprising the bone substitute and the crosslinkable prepolymer or the crosslinkable semi-IPN precursor is cured to form a cured composite.

The curable admixture and the cured composite are useful in the field of orthopedics, dentistry, and drug delivery. They can be used anywhere where bone or other tissue regeneration is required. When a therapeutic agent is incorporated in them, they are useful as drug delivery devices.

### DESCRIPTION OF THE DRAWINGS

FIG. 1A represents defects in the tibia at 8 weeks after treatment with a control.

FIG. 1B represents defects in the tibia at 8 weeks after treatment with a cured bone substitute containing Bioplant® HTR®.

FIG. 2A represents defects in the zygoma at 8 weeks after treatment with a control.

FIG. 2B represents defects in the zygoma at 8 weeks after treatment with a cured bone substitute containing Bioplant® HTR®.

### DETAILED DESCRIPTION OF THE INVENTION

The present invention relates to a polymerization initiators and cured polymers. The present invention also relates to methods of forming and using the curable admixture and cured composite.

The cured composite is formed by crosslinking the curable admixture. The curable admixture is formed by mixing an optional bone substitute and a crosslinkable prepolymer to form a substantially homogeneous mixture. The admixture can be preformed or formed immediately before application.

### Crosslinkable Anhydride Prepolymer

The crosslinkable anhydride prepolymer comprises monomers and/or oligomers having polymerizable groups, preferably radically polymerizable groups, which crosslink to form a polymer network. Suitable polymerizable groups include unsaturated alkenes (i.e., vinyl groups) such as vinyl ethers, allyl groups, unsaturated monocarboxylic acids, unsaturated dicarboxylic acids, and unsaturated tricarboxylic acids. Unsaturated monocarboxylic acids include acrylic acid, methacrylic acid, and crotonic acid. Unsaturated dicarboxylic acids include maleic, fumaric, itaconic, mesaconic or citraconic acid. The preferred polymerizable groups are acrylates, diacrylates, oligoacrylates, dimethacrylates, oligomethacrylates, and other biologically acceptable polymerizable groups. (Meth)acrylates are the most preferred active species polymerizable group.

Methacrylated sebacic acid (mSA) is one preferred methacrylate. MSA has a low viscosity and degrades rapidly. MSA is described by:

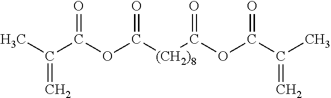

and can be synthesized according to the procedure described by Tarcha et al. J. Polym. Sci. Part A, Polym. Chem. (2001), 39, 4189. MSA is particularly useful in the present invention, particularly when additional strength is necessary. Preferably, the composition will contain a buffer when mSA is used since mSA produces acid upon degradation. The addition of mSA to the composition also provides a decreased viscosity of the pre-polymerized formulations making the prepolymer more workable. It is added to improve mechanical properties of the cured polymer.

Methacrylated carboxyphenoxyalkanes (including propane (MCPP), hexane, (MCPH) etc) are another preferred methacrylate useful in the present invention. These compounds have higher viscosity than mSA and degrade more slowly. They are also more hydrophobic than mSA. MCPP, also abbreviated as CPPDM, is (1,3-bis(carboxyphenoxy))propyl dimethacrylate:

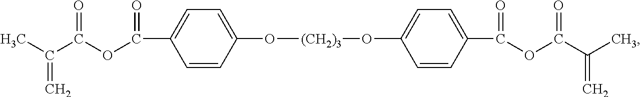

and can be synthesized according to the procedure described by Tarcha et al. J. Polym. Sci, Part A, Polym. Chem. (2001), 39, 4189.

Other polymerizable groups, including acrylates such as dimethylaminoethyl acrylate, cyanoacrylate, methyl methacrylate; N-vinyl pyrrolidone; poly(propylene fumerate); and methacrylic anhydride may also be used in a composition of the present invention.

These polymerizable groups can be present on hydrophobic or hydrophilic polymers, which can be used to adjust the hydrophobicity of the compositions. Non-limiting examples of suitable hydrophobic polymers include polyanhydrides, polyorthoesters, polyhydroxy acids, polydioxanones, polycarbonates, and polyaminocarbonates. Non-limiting examples of suitable hydrophilic polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose (and derivatives), and hydroxyalkylated celluloses (and derivatives) such as hydroxyethyl cellulose and methylhydroxypropyl cellulose, and natural polymers such as polypeptides, polysaccharides or carbohydrates such as Ficoll® polysucrose, hyaluronic acid, dextran (and derivatives), heparan sulfate, chondroitin sulfate, heparin, or alginate, and proteins such as gelatin, collagen, albumin, or ovalbumin or copolymers or blends thereof. One preferred hydrophilic polymer is dimethacrylated poly(ethylene glycol) (PEGDM). More preferably, the molecular weight of the PEGDM will be a 300 or 600. The concentration of PEGDM in the prepolymer formulation is adjusted to obtain good workability and mixing properties of the prepolymer.

Preferably, the monomer and/or oligomer comprises a biodegradable linkage such as amide-, anhydride-, carbonate-, ester-, or orthoester linkages; more preferably, an anhydride-linkage so that the polymer network formed by the monomer and/or oligomer is biodegradable.

The molecular weight of the crosslinkable prepolymer is preferably in the range of about 150 to about 20,000. Preferably, the prepolymer has from 1 to about 100 repeating units in the structure, more preferably from about 1 to about 20, and most preferably from about 1 to about 10 repeating units.

Three non-limiting embodiments of the crosslinkable prepolymer are disclosed below.

Details of First Embodiment of Crosslinkable Prepolymer

As a first preferred embodiment, the crosslinkable prepolymer is one or more anhydride monomers or oligomers. Useful monomers or oligomers include anhydrides of a diacid or multifunctional acids and carboxylic acid molecules which include a crosslinkable group such as an unsaturated moiety.

Preferably, the crosslinkable prepolymer is linear with an unsaturated hydrocarbon moiety at each terminus and comprises a dianhydride of a dicarboxylic acid monomer or oligomer and a carboxylic acid molecule comprising an unsaturated moiety. More desirably, it comprises a methacrylic acid dianhydride of a monomer or oligomer of a diacid selected from the group consisting of sebacic acid and 1,3-bis(p-carboxyphenoxy)-alkane such as 1,3-bis(p-carboxyphenoxy)-propane.

Exemplary diacids or multifunctional acids include sebacic acid (SA), 1,3-bis(p-carboxyphenoxy)-alkanes such as 1,3-bis(p-carboxyphenoxy)-propane (CPP) or 1,3-bis(p-carboxyphenoxy)-hexane (CPH), dodecanedioic acid, fumaric acid, bis(p-carboxyphenoxy)methane, terephthalic acid, isophthalic acid, p-carboxyphenoxy acetic acid, p-carboxyphenoxy valeric acid, p-carboxyphenoxy octanoic acid, or citric acid. In one embodiment, it is preferably methacrylated sebacic acid (MSA), a methacrylated 1,3-bis(p-carboxyphenoxy)-alkane (e.g., MCPP or MCPH), or a combination thereof.

Exemplary carboxylic acids include methacrylic acid, or other functionalized carboxylic acids, including, e.g., acrylic, methacrylic, vinyl and/or styryl groups. The preferred carboxylic acid is methacrylic acid.

The anhydride monomers or oligomers are formed, for example, by reacting the diacid with an activated form of the carboxylic acid, such as an anhydride thereof, to form an anhydride. A detailed description of the anhydride monomer(s) or oligomer(s) suitable as crosslinkable prepolymer(s) is provided in the '599 patent, the specification of which is incorporated by reference in its entirety.

Another route for synthesizing the methacrylated sebacic acid (MSA) and (1,3-bis(carboxyphenoxy))propyl dimethacrylate (MCPP or CPPDM) is described by Tarcha, et al., J. Polym. Sci, Part A, Polym. Chem. (2001), 39, 4189.

In a preferred embodiment, the crosslinkable prepolymer is a mixture of a first anhydride and a second anhydride. The ratio of these anhydrides can be adjusted to provide the biodegradation, hydrophilicity and/or adherence properties most suitable for a specific application.

For example, polymer networks formed by crosslinking dimethacrylated anhydride monomers formed from sebacic acid typically biodegrade much faster than that formed from 1,3-bis(p-carboxyphenoxy)-alkane(s). Hence, mixing anhydrides formed from sebacic acid with anhydrides formed from 1,3-bis(p-carboxyphenoxy)-alkane(s) in various ratios provides a wide array of degradation behaviors.

In another example, where the polymer network is formed by crosslinking 1,3-bis(p-carboxyphenoxy)-alkane(s), methacrylic anhydride is added to increase plasticity and aid in mixing. Preferably, 1-10 mol % is added. The amount of methacrylic anhydride is dependent upon the consistency of the mixture (i.e., how much of an additional agent such as PEG is incorporated) and should be sufficient to allow for adequate mixing.

The ratio of the first anhydride to the second anhydride can vary widely. Preferably, it is in the range from about 1:20 to about 20:1; more preferably from about 1:5 to about 5:1; even more preferably from about 1:5 to about 1:1, most preferably at about 1:1.

Preferably, as detailed below, the crosslinkable prepolymer comprises a photoinitiator or a combination of a photoinitiator and a redox initiator system.

Details of Second Embodiment of Crosslinkable Prepolymer

In the second embodiment, the crosslinkable prepolymer is a crosslinkable semi-IPN precursor.

The crosslinkable semi-IPN precursor comprises at least two components: the first component is a linear polymer, and the second component is one or more crosslinkable monomers or macromers. The crosslinkable semi-IPN precursor forms a semi-interpenetrating network (“semi-IPN”) when crosslinked. Semi-IPNs are defined as compositions that include two independent components, where one component is a crosslinked polymer and the other component is a non-crosslinked polymer. The crosslinkable semi-IPN precursor and the semi-IPN it forms are described in detail in U.S. Pat. No. 5,837,752 to Shastri et al., which is incorporated by reference in its entirety.

The first component of the crosslinkable semi-IPN precursor is a linear polymer. Preferably, the linear polymer in the first component is (i) a linear hydrophobic biodegradable polymer, preferably a homopolymer or copolymer which includes hydroxy acid and/or anhydride linkages, or (ii) a linear, non-biodegradable hydrophilic polymer, preferably polyethylene oxide or polyethylene glycol.

Preferably, at least one of the monomers or macromers includes a degradable linkage, preferably an anhydride linkage. The linear polymer preferably constitutes between 10 and 90% by weight of the crosslinkable semi-IPN precursor composition, more preferably between 30 and 70% of the crosslinkable semi-IPN precursor composition.

Linear polymers are homopolymers or block copolymers that are not crosslinked. Hydrophobic polymers are well known to those of skill in the art. Examples of suitable biodegradable polymers include polyanhydrides, polyorthoesters, polyhydroxy acids, polydioxanones, polycarbonates, and polyaminocarbonates. Preferred polymers are polyhydroxy acids and polyanhydrides. Polyanhydrides are the most preferred polymers.

Linear, hydrophilic polymers are well known to those of skill in the art. Examples of suitable hydrophilic non-biodegradable polymers include poly(ethylene glycol), poly(ethylene oxide), partially or fully hydrolyzed poly(vinyl alcohol), poly(ethylene oxide)-co-poly(propylene oxide) block copolymers (poloxamers and meroxapols) and poloxamines. Preferred hydrophilic non-biodegradable polymers are poly(ethylene glycol), poloxamines, poloxamers and meroxapols. Polyethylene glycol) is the most preferred hydrophilic non-biodegradable polymer.

The second component of the crosslinkable semi-IPN precursor is one or more crosslinkable monomers or macromers. Preferably, at least one of the monomers or macromers includes an anhydride linkage. Other monomers or macromers that can be used include biocompatible monomers and macromers which include at least one radically polymerizable group. For example, polymers including alkene linkages which can be crosslinked may be used, as disclosed in WO 93/17669 by the Board of Regents, University of Texas System, the disclosure of which is incorporated herein by reference.

Suitable polymerizable groups include unsaturated alkenes (i.e., vinyl groups) such as vinyl ethers, allyl groups, unsaturated monocarboxylic acids, unsaturated dicarboxylic acids, and unsaturated tricarboxylic acids. Unsaturated monocarboxylic acids include acrylic acid, methacrylic acid, and crotonic acid. Unsaturated dicarboxylic acids include maleic, fumaric, itaconic, mesaconic or citraconic acid. The preferred polymerizable groups are acrylates, diacrylates, oligoacrylates, dimethacrylates, oligomethacrylates, and other biologically acceptable polymerizable groups. (Meth)acrylates are the most preferred active species polymerizable group. In one embodiment, the preferred methacrylate is a sebacic acid (MSA), a 1,3-bis(p-carboxyphenoxy)-alkane (e.g., MCPP or MCPH), or a combination thereof.

These functional groups can be present on hydrophobic or hydrophilic polymers, which can be used to adjust the hydrophobicity of the compositions. Suitable hydrophobic polymers include polyanhydrides, polyorthoesters, polyhydroxy acids, polydioxanones, polycarbonates, and polyaminocarbonates. Suitable hydrophilic polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll® polysucrose, hyaluronic acid, dextran, heparan sulfate, chondroitin sulfate, heparin, or alginate, and proteins such as gelatin, collagen, albumin, or ovalbumin or copolymers or blends thereof.

The polymers can be biodegradable, but are preferably of low biodegradability (for predictability of dissolution) but of sufficiently low molecular weight to allow excretion. The maximum molecular weight to allow excretion in human beings (or other species in which use is intended) will vary with polymer type, but will often be about 20,000 daltons or below.

The polymers can include two or more water-soluble blocks which are joined by other groups. Such joining groups can include biodegradable linkages, polymerizable linkages, or both. For example, an unsaturated dicarboxylic acid, such as maleic, fumaric, or aconitic acid, can be esterified with hydrophilic polymers containing hydroxy groups, such as polyethylene glycols, or amidated with hydrophilic polymers containing amine groups, such as poloxamines.

Methods for the synthesis of these polymers are well known to those skilled in the art. See, for example, Concise Encyclopedia of Polymer Science and Polymeric Amines and Ammonium Salts, E. Goethals, editor (Pergamen Press, Elmsford, N.Y. 1980). Many polymers, such as poly(acrylic acid), are commercially available. Naturally occurring and synthetic polymers may be modified using chemical reactions available in the art and described, for example, in March, “Advanced Organic Chemistry,” 4th Edition, 1992, Wiley-Interscience Publication, New York.

Preferably, the monomers and/or macromers that include radically polymerizable groups include slightly more than one crosslinkable group on average per molecule, more preferably two or more polymerizable or crosslinkable groups on average per molecule. Because each polymerizable group will polymerize into a chain, crosslinked materials can be produced using only slightly more than one reactive group per polymer (i.e., about 1.02 polymerizable groups on average).

Details of Third Embodiment of Crosslinkable Prepolymer

The third embodiment of the crosslinkable prepolymer is disclosed in U.S. Pat. Pub. 2003/114552, the specification of which is hereby incorporated by reference in its entirety. Specifically, it is a crosslinkable multifunctional prepolymer comprising at least two polymerizable terminal groups and having a viscosity such that the crosslinkable prepolymer is deformable at a temperature of 0° to 60° C. into a three-dimensional shape and being crosslinkable within the temperature range. Preferably, the crosslinkable prepolymer comprises a hydrophilic region, at least one biodegradable region, and at least one polymerization region and has from 1 to about 100, more preferably from 1 to 20, most preferably 1 to 10, repeating units. The hydrophilic region preferably is a polyethylene glycol or a copolymer of ethylene oxide and an alkylene oxide with a degree of polymerization in the range of 2 to 500.

The crosslinkable prepolymer may comprise a polyacetal sequence; a polyester sequence, resulting from copolymerizing a mixture of lactones wherein none of the lactone co-monomers is present in the resulting polyester sequence in a molar proportion above 75%; or a polyorthoester sequence; or a combination of a polyester sequence and a polyorthoester sequence. The polymerizable region of the crosslinkable prepolymer contains alkenes, alkynes or both.

### Initiator System

The present invention utilizes an initiator system to cure the crosslinkable prepolymer. In one embodiment, both light curing and chemical curing is used. The initiator system is divided into two parts, an initiator and an amine accelerator. The initiator (component A) comprising the light and chemical initiators and the amine accelerator (component B) comprising the light and chemical accelerators. This system allows for fast curing of the polymer from light curing, while the chemical curing initiates the cross-linking reaction throughout the polymer matrix and increases the viscosity so that the material sets homogeneously.

In one preferred embodiment, the two components are mixed with the crosslinkable prepolymer immediately before curing. In other embodiments, one of the components is mixed with a component of the polymer or monomer or with the filler component prior to curing (e.g. to form a kit that can be easily manipulated to crosslink the prepolymer. When the initiator is pre-mixed, care must be taken to combine components so as not to degrade the polymer or prepolymer (particularly where the polymer is an anhydride which can be unstable in the presence of an oxidant) or destroy the initiator.

Initiator—Component A

In a first embodiment, Component A comprises a light radical generating component activated by electromagnetic radiation, i.e., a photoinitiator. This may be ultraviolet light (e.g., long wavelength ultraviolet light), light in the visible region, focused laser light, infra-red and near-infra-red light, X-ray radiation or gamma radiation. Preferably, the radiation is light in the visible region and, more preferably, is blue light. Exposure of the photoinitiator and a co-catalyst such as an amine to light generates active species. Light absorption by the photoinitiator causes it to assume a triplet state; the triplet state subsequently reacts with the co-catalyst to form an active species which initiates polymerization.

Non-limiting examples of the photoinitiators include biocompatible photoinitiators such as beta carotene, riboflavin, Irgacure 651® (2,2-dimethoxy-2-phenylacetophenone), phenylglycine, dyes such as erythrosin, phloxime, rose bengal, thonine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, and other acetophenone derivatives, and camphorquinone. A preferred photoinitiator is camphorquinone.

Component A also comprises a second free radical generator (i.e., a chemical radical generator). The free radical generator is an oxidizing agent (also called an oxidizing component), such as peroxide. This agent is combined in a redox couple by mixing component A with component B, resulting in the generation of an initiating species (such as free radicals, anions, or cations) capable of causing curing. Preferably, the redox couples of this invention are activated at temperatures below about 40° C., for example, at room temperature or at the physiological temperature of about 37° C. The redox couple is partitioned into separate reactive components A and B prior to use and then subsequently mixed at the time of use to generate the desired initiating species. Selection of the redox couple is governed by several criteria. For example, a desirable oxidizing agent is one that is sufficiently oxidizing in nature to oxidize the reducing agent, but not excessively oxidizing that it may prematurely react with other components with which it may be combined during storage. Oxidation of the resin with an inappropriate oxidizing agent could result in an unstable system that would prematurely polymerize and subsequently provide a limited shelf life.

Suitable oxidizing agents include peroxide compounds (i.e., peroxy compounds), including hydrogen peroxide as well as inorganic and organic peroxide compounds (e.g., “per” compounds or salts with peroxoanions). Examples of suitable oxidizing agents include, but are not limited to: peroxides such as benzoyl peroxide, phthaloyl peroxide, substituted benzoyl peroxides, acetyl peroxide, caproyl peroxide, lauroyl peroxide, cinnamoyl peroxide, acetyl benzoyl peroxide, methyl ethyl ketone peroxide, sodium peroxide, hydrogen peroxide, di-tert butyl peroxide, tetraline peroxide, urea peroxide, and cumene peroxide; hydroperoxides such as p-methane hydroperoxide, di-isopropyl-benzene hydroperoxide, tert-butyl hydroperoxide, methyl ethyl ketone hydroperoxide, and 1-hydroxy cyclohexyl hydroperoxide-1, ammonium persulfate, sodium perborate, sodium perchlorate, potassium persulfate, ozone, ozonides, 2-hydroxy-4-methoxy-benzophenone, 2 (2-hydroxy-5-methylphenyl)benzotriazol etc. Benzoyl peroxide is the preferred oxidizing agent. Other oxidizing agents include azo initiators, such as azoisobutyronitrile (AIBN) or 2,2-azobis(2-amidopropane)dihydrochloride.

These oxidizing agents may be used alone or in admixture with one another. One or more oxidizing agents may be present in an amount sufficient to provide initiation of the curing process. Preferably, this includes about 0.01 weight percent (wt-%) to about 4.0 wt-%, and more preferably about 0.05 wt-% to about 1.0 wt-%, based on the total weight of all components of the dental material.

Thus, suitable redox couples individually provide good shelf-life (for example, at least 2 months, preferably at least 4 months, and more preferably at least 6 months in an environment of 5-20° C.), and then, when combined together, generate the desired initiating species for curing or partially curing the curable admixture. The shelf life of the photoinitiator is dependent on the exposure to light. It is therefore preferred to store component A in an opaque container and/or in the dark. It is also preferred to formulate A such that oxidizers in the formulation do not react with the other components in the mixture and thereby reduce the shelf life.

In one particular embodiment, component A contains camphorquinone (CQ) and benzoyl peroxide (BPO). Preferably, the relative amounts (w/w) are between 5:1 and 1:5, more preferably between 2:1 and 1:2, and desirably about 1:1.

The light and chemical radical generating components are preferably dissolved in a liquid such as a PEG, PEG methacrylate, or a PEG dimethacrylate. Ethyl acetate, acetone, N-methyl-pyrrolidone, and/or N-vinyl pyrrolidone may also be added. The liquid primarily acts as a solvent for the initiator component and can be selected dependent on the viscosity desired for the mixture. Some of the solvents will also polymerize upon curing, and be incorporated into the polymer matrix (i.e., a reactive polymer). It may contain a reactive or non-reactive polymer that can be both a solvent and part of the shell polymer matrix. In addition to being a solvent, the liquid may also be used as a pore-generating agent (i.e., as the solvent evaporates, it leaves voids, or pores), or the liquid may have additional functionality.

When making component A, the order of mixing can be important to retain solubility and activity of the component. For example, in an embodiment containing CQ and BPO in a PEG and ethyl acetate mixture, the ethyl acetate should be mixed with the CQ and BPO before the PEG is added. It is also beneficial to obtain homogeneity in component A to obtain a good polymer cure.

In a second embodiment, Component A contains a chemical radical generating component but no light radical generating component.

Amine Accelerator—Component B

In a first embodiment, Component B comprises a light accelerator component (or co-catalyst) and a reducing agent. Exposure of the photoinitiator to light generates a triplet state which reacts with the light accelerator co-catalyst component to form an active species that initiates polymerization. Preferred co-catalysts are amines, and more particularly the aromatic amines. Examples of aromatic amine accelerators include: N-alkyl substituted alkylamino benzoates, such as 4-ethyl-dimethyl amino benzoate (4-EDMAB); benzylamines such as N,N-dimethylbenzylamine and N-isopropylbenzylamine; dibenzyl amine; 4-tolyldiethanolamine; and N-benzylethanolamine. Additionally, other suitable amine accelerators include N-alkyldiethanolamines such as N-methyldiethanolamine; triethanolamine; and triethylamine. One particularly preferred aromatic amine is 4-EDMAB.

The reducing agent, which is also called a chemical accelerator, is also in component B. A desirable reducing agent is one that is sufficiently reducing in nature to readily react with the preferred oxidizing agent, but not excessively reducing in nature such that it may reduce other components with which it may be combined during storage. Reduction of the resin with an inappropriate reducing agent could result in an unstable system that would prematurely polymerize and subsequently provide a limited shelf life.

A reducing agent has one or more functional groups for activation of the oxidizing agent. Preferably, such functional group(s) is selected from amines, mercaptans, or mixtures thereof. If more than one functional group is present, they may be part of the same compound or provided by different compounds. A preferred reducing agent is a tertiary aromatic amine (e.g., N,N-dimethyl-p-toluidine (DMPT) or N,N-bis(2-hydroxyethyl)-p-toluidine (DHEPT)). Examples of such tertiary amines are well known in the art and are disclosed, for example, in WO 97/35916 and U.S. Pat. No. 6,624,211. Another preferred reducing agent is a mercaptan, which can include aromatic and/or aliphatic groups, and optionally polymerizable groups. Preferred mercaptans have a molecular weight greater than about 200 as these mercaptans have less intense odor. Other reducing agents, such as sulfinic acids, formic acid, ascorbic acid, hydrazines, some alcohols, and salts thereof, can also be used herein to initiate free radical polymerization.

If two or more reducing agents are used, they are preferably chosen such that at least one has a faster rate of activation than the other(s). That is, one causes a faster rate of initiation of the curing of the curable admixture than the other(s).

Electrochemical oxidation potentials of reducing agents and reduction potentials of oxidizing agents are useful tools for predicting the effectiveness of a suitable redox couple. For example, the reduction potential of the oxidant (i.e., oxidizing agent) benzoyl peroxide is approximately −0.16 volts vs. a saturated calomel electrode (SCE). Similarly, the oxidation potential (vs. SCE) for a series of amines has been previously established as follows: (e.g., N,N-dimethyl-p-toluidine ((DMPT), 0.61 volt), dihydroxyethyl-p-toluidine ((DHEPT), 0.76 volt), 4-t-butyl dimethylaniline ((t-BDMA), 0.77 volt), 4-dimethylaminophenethanol ((DMAPE), 0.78 volt), triethylamine ((TEA, 0.88 volt), 3-dimethylaminobenzoic acid ((3-DMAB) 0.93 volt), 4-dimethylaminobenzoic acid ((4-DMAB, 1.07 volts), ethyl p-dimethylaminobenzoate ((EDMAB), 1.07 volts), 2-ethylhexyl p-dimethylaminobenzoate ((EHDMAB), 1.09 volts) and 4-dimethylaminobenzoate ((DMABA), 1.15 volts). The ease of oxidation (and subsequent reactivity) increases as the magnitude of the oxidation decreases. Suitable amine reducing agents in combination with benzoyl peroxide generally include aromatic amines with reduction potentials less than about 1.00 volt vs. SCE. Less effective oxidants than benzoyl peroxide such as lauroyl peroxide (reduction potential=−0.60 volt) are poorer oxidizing agents and subsequently react more slowly with aromatic amine reducing agents. Suitable aromatic amines for lauroyl peroxide will generally include those having reduction potentials less than about 0.80 volt vs. SCE.

A preferred reducing agent is N,N-dimethyl-p-toluidine (DMT, also known as DMPT). When DMT is used, its percentage is preferably kept low to reduce heating of the sample that occurs during curing. It is preferred to keep the temperature below about 50° C. for the entire mixing process. In one particular exemplary embodiment, component B comprises 4-EDMAB and DMT in a ratio between 2:1 and 1:2.

In one embodiment, it is contemplated that a single agent (i.e., DMT) can be both the reducing agent and light accelerator of component B. This molecule must both have a suitable oxidation potential with the oxidizing agent and interact with the triplet state of the photoinitiator. In this embodiment, no other agent is required in component B.

It is also contemplated that instead of an oxidizing agent in component A and reducing agent in component B, component A will contain a reducing agent and component B will contain the oxidizing agent. For this embodiment, the selection of the redox couple must be done with care so as not to provide a reducing agent that can act as an accelerator or otherwise react with the photoinitiator before the crosslinking is initiated by mixing the components.

In a second embodiment, the present invention comprises an initiator system having only a chemical curing component. This initiator system is also divided into two parts, the first part (component A) comprising the chemical initiator and the second part (component B) comprises the chemical accelerator as discussed above.

Additional Initiators

Other initiators may also be added to the formulations of the present invention. Such initiators include additional photoinitiators or redox initiators. They also include thermal initiators, including peroxydicarbonate, persulfate (e.g., potassium persulfate or ammonium persulfate). Thermally activated initiators, alone or in combination with other type of initiators, are most useful where light can not reach (e.g., deep within the curable admixture). Additionally, multifunctional initiators may be used. These initiators may be added into component A or component B such that the initiator will not react with the other ingredients in component A or B before the component is mixed with the monomer, polymer, or other component.

### Fillers

The curable admixture and/or cured composite of the present invention may contain the following optional fillers. These fillers, such as a bone substitutes may be incorporated into the polymer of the present invention. The filler, such as a bone substitute bone substituted is added when increased strength and/or slow resorption is required. The ratio of the bone substitute to crosslinkable prepolymer in the curable admixture may be a wide range of values. Preferably, the ratio is from 1:20 to 20:1; more preferably from 1:4 to 1:1; most preferably from about at about 1:2 to 1:1. The bone substitute can be any bone graft material known to one skilled in the art, preferably a ceramic or a polymer. Examples include Bioplant® HTR®, HA, TCP, and combinations thereof. It can be organic or synthetic or a combination thereof. Organic bone substitutes include autograft, allograft, xenograft or combinations thereof. Cadaver-derived materials and bovine-derived materials are non-limiting examples of allografts. Bovine-derived materials (e.g., Osteograft® N-300 and Osteograft® N-700) are non-limiting examples of xenografts. Synthetic bone substitutes are also known as alloplasts. Non-limiting examples of the alloplast include calcium phosphate and calcium sulfate ceramics and polymeric bone graft materials. In one embodiment, the bone substitute comprises an alloplast, more preferably a polymeric alloplast. The bone substitute may also be a polymer-ceramic hybrid, which is combination of a polymer material and a ceramic material mixed or combined to provide preferable properties of hardness, porosity, and resorbability.

Acrylic Polymers (BIOPLANT® HTR®)

In one embodiment, the polymeric alloplast is preferably a plurality of micron-sized particles (preferably with a diameter from about 250 to 900 microns), each particle comprising a core layer comprised of a first polymeric material and a coating generally surrounding the core layer. The coating comprises a second polymeric material which is hydrophilic and has a composition different from the composition of the first polymeric material. Both polymeric materials in the polymeric alloplast are biocompatible. The first polymeric material is preferably an acrylic polymer and more preferably poly(methyl methacrylate) (PMMA). The PMMA may further include a plasticizer, if desired. The second polymeric material is preferably a polymeric hydroxyethyl methacrylate (PHEMA). Preferred polymeric particles are disclosed in the '485 patent, the specification of which is hereby incorporated by reference in its entirety.

In a more preferred embodiment, the bone substitute is a plurality of calcium hydroxide-treated polymeric micron-sized particles. The quantity of calcium hydroxide is effective to induce the growth of hard tissue in the pores and on the surface of the polymeric micron-sized particles when packed in a body cavity. Preferably, the calcium hydroxide forms a coating on both the outer and inner surfaces of the polymeric particles.

The micron-sized particles of the bone substitute may further optionally include a non-bonding agent, such as barium sulfate, to prevent the particles from bonding together. Barium sulfate is also a radio-opaque compound and may be included so as to render the curable admixture and the cured composite visible on an X-ray radiograph. The calcium hydroxide also assists in preventing the polymeric particles from bonding together.

Preferred procedures for producing the bone substitute component of the curable admixture of the present invention are set forth in the specification of the '158 patent. Preferably, calcium hydroxide is introduced into the pores of the micron-sized particles by soaking the particles in an aqueous solution of calcium hydroxide, then removing any excess solution from the particles and allowing the particles to dry. Preferred aqueous solutions of calcium hydroxide have a concentration in the range of from about 0.05 percent to about 1.0 percent calcium hydroxide by weight.

In a most preferred embodiment, the bone substitute is Bioplant® HTR,® available from Bioplant Inc. (Norwalk, Conn.), set forth in the '570 patent, which is hereby incorporated by reference in its entirety. The Bioplant® HTR® are microporous particles of calcified (Ca(OH)₂/calcium-carbonate) copolymer of PMMA and PHEMA, with the outer calcium layer interfacing with bone forming calcium carbonate-apatite. The outer diameter of the particles is about 750 μm; the inner diameter is about 600 μm and the pore opening diameter is about 350 μm. Bioplant® HTR® is strong (forces greater than 50,000 lb/in will not crush the Bioplant® HTR® particles), biocompatible and negatively charged (−10 mV) to promote cellular attraction and resist infection. In another embodiment, a smaller particle size Bioplant® HTR® is used, having an outer diameter of 200-400 μm. This smaller diameter Bioplant® HTR® could be more beneficial for injectable formulations where an ability to flow through a syringe is important.

Bioplant® HTR® is added to the composition of the present invention from 0-60% w/w. In one preferred embodiment, 30-50% Bioplant® HTR® will be added to the composition. This relatively large amount of Bioplant® HTR® provides the composition with a surface having a preferred surface composition for promoting new bone growth. In another embodiment, 20-30% Bioplant® HTR® is added to the composition.

Hydroxyapatite (HA) and Tricalcium Phosphate (TCP)

In one embodiment, the polymeric alloplast is preferably a hydroxyapatite (HA) filler. Hydroxyapatite, (Ca₁₀(PO₄)₆(OH)₂) is one of the most biocompatible materials with bones; it is naturally found in bone mineral and in the matrix of teeth and provides rigidity to bones and teeth. When a HA-containing material is used as a filler in the present invention, the modulus will be significantly increase.

A non-limiting list of HA bone substitute, or filler compounds that may be used in the present invention include: Pro Osteon® (Interpore Cross International, Inc., Irvine, Calif.) comprising monolithic ceramic granules, which are made using coralline calcium carbonate fully or partially converted to HA by a hydrothermal reaction, see D. M. Roy and S. K. Linnehan, Nature, 247, 220-222 (1974); R. Holmes, V. Mooney, R. Bucholz and A. Tencer, Clin. Orthop. Rel. Res., 188, 252-262 (1984); and W. R. Walsh, et al., J. Orthop. Res., 21, 4, 655-661 (2003). VITOSS® (Orthovita, Malvern, Pa.) is provided as monolithic ceramic granules. Norian SRS® (Synthes-Stratec, affiliates across Europe and Latin America) and Alpha-BSM® (ETEX Corp., Cambridge, Mass.) are provided as an injectable pastes. ApaPore® and Pore-SI (ApaTech, London, England) are currently under development and comprise monolithic ceramic granules.

In one embodiment, the filler is preferably a material based upon HA, including the resorbable carbonated apatite. One particularly preferred HA, is a porous calcium phosphate material which is a porous hydroxyapatite and is more integrable, absorbable and more osteoconductive than dense hydroxyapatite. Porous HA can be made by the methods described in EP1411035, herein incorporated by reference. The aporosity can be controlled both as a ratio of the volume of material to the volume of air and as the porosity and pore size distribution.

Additionally, recent studies have elucidated the detrimental and beneficial effects of minor amounts of impurities and some dopants. Parts per million levels of lead, arsenic, and the like, if incorporated into hydroxyapatite, may lead to inhibition of osteoconduction. It is therefore preferable to use HA substantially free from these impurities. On the other hand, carbonated apatite exhibits faster bioresorption than pure HA, if desired, and 1-3 wt % silicon additions to HA have shown a two-fold increase in the rate of osteoconduction over pure HA, see N. Patel, et al., J. Mater. Sci: Mater. Med., 13, 1199-206 (2002); and A. E. Portera, et al., Biomaterials, 24, 4609-4620 (2002). Silicon-doped HA such as the doped HA being developed at ApaTech and may be used as a filler in the present invention.

The HA is added to the composition of the present invention from 0-60% w/w. In one preferred embodiment, 20-30% HA is added to the composition.

In one embodiment, the filler is preferably a calcium phosphate material based upon HA, including alpha (α-TCP) or beta-tricalcium phosphate (Ca₃(PO₄)₂, α-TCP), which is a close synthetic equivalent to the composition of human bone mineral and has favorable resorption characteristics.

α-TCP has a high resorbability when the material is implanted in a bone defect and is sold as Biosorb®. Other calcium phosphates including biphasic calcium phosphate or BCP (an intimate mixture of HA and α-TCP) and unsintered apatite (AP) may also be used as bone substitutes in the present invention.

In another embodiment, the TCP material may be a TCP having a particularly small crystal size and/or particle size. This TCP (i.e., α- and/or β-TCP) is formed into high surface area powders, coatings, porous bodies, and dense articles by a wet chemical approach and transformed into TCP, for example by a calcination step such as that described in U.S. Pat. Pub. 2005/0031704, herein incorporated by reference. This TCP material, generally having an average TCP crystal size of about 250 nm or less and an average particle size of about 5 μm or less, has greater reliability and better mechanical properties as compared to conventional TCP having a coarser microstructure and is therefore one particularly preferred embodiment of the present invention.

Calcium

Ca(OH)₂, or CaCO₃ provides a good source of calcium for bone formation, it also provides a polymer surface that promotes bone growth. Additionally, the calcium will neutralize the pH of the polymer. This is particularly relevant when mSA is included in the formulation since this acid will alter the pH upon degradation. Non-limiting examples of compounds providing calcium including Ca(OH)₂, or CaCO₃, demineralized bone powder or particles, coral powder, calcium phosphate particles, α-tricalcium phosphate, octacalcium phosphate, calcium carbonate, and calcium sulfate. Preferably, such calcium compounds can neutralize the acid generated during the degradation of a biodegradable polymer and maintain a physiological pH value suitable for bone formation. It is preferably alkaline in nature so that it can neutralize the acid generated in the biodegradation process and help to maintain a physiological pH value.

Linear Polymers

Additional fillers such as a linear polyamide (PA), polyglycolic acid (PGA), polylactide (PLA), or a PGA/PLA copolymer can be added, for example, to reduce or eliminate shrinkage. For example, 1-25% of a linear PA may be used in a composition having 80% MCPP and 20% MSA. Greater amounts are generally not indicated due to a potential reduction in the consistency of the composition.

Other linear polymers are copolymers such as poly(CPH-SA) and poly(CPP-SA). These non-reactive polyanhydride copolymers may be added as an additional filler.

### Additional Agents

One or more additional agents may also be added to the composition, dependant upon the intended use.

Inhibitors

Inhibitors may also be added to the formulation. Inhibitors can be used to prolong the shelf life of the individual components before curing the polymer system. A non-limiting list of inhibitors that may be added to the polymeric compositions of the present invention include phenols such as hydroquinone, mono methyl hydroquinone, and 2,6-bitertbutyl-4-methyl phenol; vitamin E; 4-text butyl catechal; and aliphatic and aromatic amines such as phenylenediamines.

Excipients

One or more excipients may be incorporated into the compositions of the present invention.

Steric acid is a preferred excipient. Steric acid is non-reactive and acts as a diluent. It can be used to increase hydrophobicity, reduce strength, and increase consistency of the polymer formulation.

Ethyl acetate is another excipient that may be used to aid in the salvation and mixing as well as to obtain a viscosity useful for working with the polymerizable material.

Porosity Forming Agents

One or more substances that promote pore formation may be incorporated into the composition of the present invention; preferably in the curable composite.

Non-limiting examples of such substances include: particles of inorganic salts such as NaCl, CaCl₂, porous gelatin, carbohydrate (e.g., monosaccharide), oligosaccharide (e.g., lactose), polysaccharide (e.g., a polyglucoside such as dextran), gelatin derivative containing polymerizable side groups, porous polymeric particles, waxes, such as paraffin, bees wax, and carnaba wax, and wax-like substances, such as low melting or high melting low density polyethylene (LDPE), and petroleum jelly. Other useful materials include hydrophilic materials such as PEG, alginate, bone wax (fatty acid dimers), fatty acid esters such as mono-, di-, and tri-glycerides, cholesterol and cholesterol esters, and naphthalene. In addition, synthetic or biological polymeric materials such as proteins can be used.

The size or size distribution of the porosity forming agent particles used in the invention can vary according to the specific need. Preferably the particle size is less than about 5000 μm, more preferably between about 500 and about 5000 μm, even more preferably between about 25 and about 500 μm, and most desirably between about 100 and 250 μm.

Bone Promoting Agents

One or more substances that promote and/or induce bone formation may be incorporated into the compositions of the present invention. The bone promoting agent can include, for example, proteins originating from various animals including humans, microorganisms and plants, as well as those produced by chemical synthesis and using genetic engineering techniques. Such agents include, but are not limited to, biologically active substances such as growth factors such as, bFGF (basic fibroblast growth factor), acidic fibroblast growth factor (aFGF) EGF (epidermal growth factor), PDGF (platelet-derived growth factor), IGF (insulin-like growth factor), the TGF-β superfamily (including TGF-β s, activins, inhibins, growth and differentiation factors (GDFs), and bone morphogenetic proteins (BMPs)), cytokines, such as various interferons, including interferon-α, -β, and γ, and interleukin-2 and -3; hormones, such as, insulin, growth hormone-releasing factor and calcitonin; non-peptide hormones; antibiotics; chemical agents such as chemical mimetics of growth factors or growth factor receptors, and gene and DNA constructs, including cDNA constructs and genomic constructs. In a preferred embodiment, the agents include those factors, proteinaceous or otherwise, which are found to play a role in the induction or conduction of growth of bone, ligaments, cartilage or other tissues associated with bone or joints, such as for example, BMP and bFGF. The present invention also encompasses the use of autologous or allogeneic cells encapsulated within the composition. The autologous cells may be those naturally occurring in the donor or cells that have been recombinantly modified to contain nucleic acid encoding desired protein products.

Non-limiting examples of suitable bone promoting materials include growth factors such as BMP (Sulzer Orthopedics), BMP-2 (Medtronic/Sofamor Danek), bFGF (Orquest/Anika Therapeutics), Epogen (Amgen), granulocyte colony-stimulating factor (G-CSF) (Amgen), Interleukin growth factor (IGF)-1 (Celtrix Pharmaceuticals), osteogenic protein (OP)-1 (Creative BioMolecules/Stryker Biotec), platelet-derived growth factor (PDGF) (Chiron), stem cell proliferation factor (SCPF) (University of Florida/Advanced Tissue Sciences), recombinant human interleukin (rhIL) (Genetics Institute), transforming growth factor beta (TGFβ) (Collagen Corporation/Zimmer Integra Life Sciences), and TGFβ-3 (OSI Pharmaceuticals). Bone formation may be reduced from several months to several weeks. In orthopedic and dental applications, bone regenerating molecules, seeding cells, and/or tissue can be incorporated into the compositions. For example bone morphogenic proteins such as those described in U.S. Pat. No. 5,011,691, the disclosure of which is incorporated herein by reference, can be used in these applications.

In one embodiment, the addition of a TGF-β superfamily member is particularly preferred. These proteins are expressed during bone and joint formation and have been implicated as endogenous regulators of skeletal development. They are also able to induce ectopic bone and cartilage formation and play a role in joint and cartilage development (Storm E E, Kingsley D M. Dev Biol. 1999 May 1; 209 (1):11-27; Shimaoka et al., J Biomed Mater Res A. 200468 (1):168-76; Lee et al., J Periodontol. 2003 74 (6):865-72). The BMP proteins that may be used include, but are not limited to BMP-1 or a protein from one of the three subfamilies. BMP-2 (and the recombinant form rhBMP2) and BMP-4 have 80% amino acid sequence homology. BMP-5, -6, and -7 have 78% % amino acid sequence homology. BMP-3 is in a subfamily of its own. Normal bone contains approximately 0.002 mg BMP/kg bone. For BMP addition to induce bone growth at an osseous defect, 3 to 3.5 mg BMP has been found to be sufficient, although this number may vary depending upon the size of the defect and the length of time it will take for the BMP to release. Additional carriers for the BMP may be added, and include, for example, inorganic salts such as a calcium phosphate or CaO4S. (Rengachary, S S., Neurosurg. Focus, 13 (6), 2 (2002)). Particular GDFs useful in the present invention include, but are not limited to GDF-1; GDF-3 (also known as Vgr-2); the subgroup of related factors: GDF-5, GDF-6, and GDF-7; GDF-8 and highly related GDF-11; GDF-9 and -9B; GDF-10; and GDF-15 (also known as prostate-derived factor and placental bone morphogenetic protein).

It is important for the bone promoting agent to remain active through the polymerization process. For example, many enzymes, cytokines, etc. are sensitive to the radiation used to cure polymers during photopolymerization. The method provided in Baroli et al., J. Pharmaceutical Sci. 92:6 1186-1195 (2003) can be used to protect sensitive molecules from light-induced polymerization. This method provides protection using a gelatin-based wet granulation. This technique may be used to protect the bone promoting agent incorporated into the polymer composition.

Therapeutic Agents

One or more preventive or therapeutic active agents and salts or esters thereof may be incorporated into the compositions of the present invention, including but not limited to:

antipyretic analgesic anti-inflammatory agents, including non-steroidal anti-inflammatory drugs (NSAIDs) such as indomethacin, aspirin, diclofenac sodium, ketoprofen, ibuprofen, mefenamic acid, azulene, phenacetin, isopropylantipyrin, acetaminophen, benzydamine hydrochloride, phenylbutazone, flufenamic acid, mefenamic acid, sodium salicylate, choline salicylate, sasapyrine, clofezone or etodolac; and steroidal drugs such as dexamethasone, dexamethasone sodium sulfate, hydrocortisone, or prednisolone;

antibacterial and antifungal agents such as penicillin, ampicillin, amoxicillin, cephalexin, erythromycin ethylsuccinate, bacampicillin hydrochloride, minocycline hydrochloride, chloramphenicol, tetracycline, erythromycin, fluconazole, itraconazole, ketoconazole, miconazole, terbinafine; nlidixic acid, piromidic acid, pipemidic acid trihydrate, enoxacin, cinoxacin, ofloxacin, norfloxacin, ciprofloxacin hydrochloride, sulfamethoxazole, or trimethoprim;

anti-viral agents such as trisodium phosphonoformate, didanosine, dideoxycytidine, azido-deoxythymidine, didehydro-deoxythymidine, adefovir dipivoxil, abacavir, amprenavir, delavirdine, efavirenz, indinavir, lamivudine, nelfinavir, nevirapine, ritonavir, saquinavir or stavudine;

high potency analgesics such as codeine, dihydrocodeine, hydrocodone, morphine, dilandid, demoral, fentanyl, pentazocine, oxycodone, pentazocine or propoxyphene; and

salicylates which can be used to treat heart conditions or as an anti-inflammatory.

The agents can be incorporated in the composition of the invention directly, or can be incorporated in microparticles which are then incorporated in the composition. Incorporating the agents in microparticles can be advantageous for those agents, which are reactive with one or more of the components of the composition.

The method described in Baroli et al., J. Pharmaceutical Sci. 92:6 1186-1195 (2003) can be used to protect sensitive therapeutic agents from light-induced polymerization when incorporated in the polymer composition.

Diagnostic Agents

One or more diagnostic agents may be incorporated into the compositions of the present invention. Diagnostic/imaging agents can be used which allow one to monitor bone repair following implantation of the compositions in a patient. Suitable agents include commercially available agents used in positron emission tomography (PET), computer assisted tomography (CAT), single photon emission computerized tomography, X-ray, fluoroscopy, and magnetic resonance imaging (MRI).

Examples of suitable agents useful in MRI include the gadolinium chelates currently available, such as diethylene triamine pentaacetic acid (DTPA) and gadopentotate dimeglumine, as well as iron, magnesium, manganese, copper and chromium gadolinium chelates.

Examples of suitable agents useful for CAT and X-rays include iodine based materials, such as ionic monomers typified by diatrizoate and iothalamate, non-ionic monomers such as iopamidol, isohexyl, and ioversol, non-ionic dimers, such as iotrol and iodixanol, and ionic dimers, for example, ioxagalte.

These agents can be detected using standard techniques available in the art and commercially available equipment.

### Crosslinking the Curable Admixture to Form the Cured Composite

The curable admixture is crosslinked through the use of component A and B and, when photoinitiation is used, light to form the cured composite. The components are mixed thoroughly with the polymer or prepolymer(s). A ball mixer may be used to improve the consistency of mixing.

It is important to keep component A separated from component B before initiating polymerization so that the materials within the two components do not react or cure before the polymerization reaction is started. In some instances, it is similarly important to keep component A separated from the polymers or polymerizable material before use since the photochemical initiator can initiate at least some polymerization even without the accelerator component.

The concentration of the initiator(s) used is dependant on a number of factors. Non-limiting examples of such factors include the type of the initiator, whether the initiator is used alone or in combination with other initiators, the desirable rate of curing, and how the material is applied. The concentration of each initiator is between about 0.05% (w/w) to about 5% (w/w) of the crosslinkable prepolymer. Preferably, the concentration is less than 1% (w/w) of the crosslinkable prepolymer; more preferably between 0.01 and 0.1% (w/w). In one embodiment, 20 μl of component A (0.5/ml total initiators) and 20 μl of component B (0.4 g/ml total initiators) are added per gram of polymer. In another embodiment, 40 μl of each component is added per gram of polymer to effect a stronger polymer.

It is preferred to utilize a particular sequence of adding the initiator components A and B, since mixing in any other order could drastically reduce the amount or homogeneity of the polymerization reaction. In one illustrative embodiment, component A is mixed with the polymer or prepolymer until evenly dispersed. Next, component B is mixed into the composition. If the mixing of component B was rapid, the mixture should be allowed to stand for about 10-30 seconds (with optional occasional mixing). The viscosity of the mixture should noticeably increase. At this point, it is possible to transfer into a mold or inject into a space in which the polymerization should occur. Light is then directed onto the sample for 0.5, 1, 2, 3, or more minutes to complete curing. The light may, for example, be a UV, white, or blue light. A dental blue light (e.g., a Demitron or a 3M light) may be used. Most of the photo-initiated curing should occur within one minute, however, longer exposure to the light is also acceptable.

Samples of up to 1.5 cm have been cured in this manner. It is possible to cure thicker samples that are less opaque or where the chemical curing provides substantially more of the cure in the sample section farther from the light source. The size and shape of the sample is a factor in the curing of the polymer; thicker samples will take longer to cure. Additionally, larger samples may not receive the same exposure to the light source across the sample surface due to the size of the source and variations in light intensity. Since many light sources have a Gaussian profile, it may be advisable to move either the sample or the light source across the sample surface during curing to effect an evenly cured composite.

In the embodiments of the present invention where only chemical curing is used, components A and B will contain the redox components but not the photocuring agents. In one such preferred embodiment, in which component A contains benzoyl peroxide and component B contains DMT, these can be combined to initiate curing in a molar ratio of approximately 1:1. The same initiator concentration as used for combined light and chemical curing may be used for chemical-only curing, and is preferably below 1%.

In one embodiment, the crosslinkable monomer or polymer and initiator B are combined prior to use. Initiator component B may contain a photo initiator and a redox agent, just a redox agent, or an agent that is effective as both a photo initiator and a redox agent. This mixture is mixed with initiator component A when the composite material is needed, forming a simple two-phase system. The material is then packed in the bone cavity or other area, and light is directed onto the mixture to initiate polymerization if applicable.

In another embodiment, the crosslinkable monomer or polymer and initiator Component A are combined prior to use. This mixture is mixed with initiator component B when the composite material is needed, forming a simple two-phase system. The material is then packed in the bone cavity or other area, and light is directed onto the mixture to initiate polymerization if applicable.

The crosslinkable bone substitute is subjected to electromagnetic radiation from a radiation source for a period sufficient to crosslink the bone substitute and form a crosslinked composite. Preferably, the crosslinkable bone substitute is applied in layer(s) of 1-10 mm, more preferably about 3-5 mm, and subjected to an electromagnetic radiation for about 30 to 300 seconds, preferably for about 50 to 100 seconds, and more preferably for about 60 seconds.

Typically, a minimum of 0.01 mW/cm² intensity is needed to induce polymerization. Maximum light intensity can range from 1 to 1000 mW/cm², depending upon the wavelength of radiation. Tissues can be exposed to higher light intensities, for example, to longer wavelength visible light, which causes less tissue/cell damage than shortwave UV light. In dental applications, blue light is used at intensities of 100 to 400 mW/cm² clinically. When UV light is used in situ, it is preferred that the light intensity is kept below 20 mW/cm².

In another embodiment, when a thermally activated initiator is used, the crosslinkable bone substitute is subjected to a temperature suitable for activating the thermally activated initiators, preferably at a temperature from about 20 to 80° C., more preferably from about 30 to 60° C. Heat required to activate the thermal activator can be generated by various known means, including but not limited to infrared, water bath, oil bath, microwave, ultrasound, or mechanical means. For example, one can place the bone substitute in a crucible heated by a hot water bath.

In yet another embodiment, when a redox initiator system is used (alone or in combination with other type(s) of initiator(s)), the oxidizing agent of the redox initiator system is kept apart from the reducing agent of the redox initiator system until immediately before the curing process. For example, the oxidizing agent is mixed with some crosslinkable bone substitute in one container and the reducing agent is also mixed with some crosslinkable bone substitute in another container. The contents of the two containers are mixed with each other at which point substantial crosslinking is initiated.

In a most preferred embodiment, in order to shorten the duration of the radiation exposure and/or increase the thickness of the radiation crosslinkable layer, a redox initiator system is used in combination with a photoinitiator and/or thermal initiator. For example, the redox initiator system is activated first to partially crosslink the crosslinkable bone substitute. Such partially crosslinked bone substitute is then subjected to radiation and the photoinitiator and/or thermal initiator is activated to further crosslink the partially crosslinked admixture.

As used herein: “Electromagnetic radiation” refers to energy waves of the electromagnetic spectrum including, but not limited to, X-ray, ultraviolet, visible, infrared, far infrared, microwave, radio-frequency, sound and ultrasound waves. “X-ray” refers to energy waves having a wavelength of 1×10⁻⁹ to 1×10⁻⁶ cm. “Ultraviolet light” refers to energy waves having a wavelength of at least approximately 1.0×10⁻⁶ cm but less than 4.0×10⁵ cm. “Visible light” refers to energy waves having a wavelength of at least approximately 4.0×10⁻⁵ cm to about 7.0×10⁻⁵ cm. “Blue light” refers to energy waves having a wavelength of at least approximately 4.2×10⁻⁵ cm but less than 4.9×10⁻⁵ cm. “Red light” refers to energy waves having a wavelength of at least approximately 6.5×10⁻⁵ cm but less than 7.0×10⁻⁵ cm. “Infrared” refers to energy waves having a wavelength of at least approximately 7.0×10⁻⁵ cm.

Audible sound waves are in frequency ranges from 20 to 20,000 Hz. Infrasonic waves are in frequency ranges below 20 Hz. Ultrasonic waves are in frequency ranges above 20,000 Hz. “Radiation source” as used herein refers to a source of electromagnetic radiation. Examples include, but are not limited to, lamps, the sun, blue lamps, and ultraviolet lamps.

The consistence of the compositions of the present invention before curing can be varied, depending upon the intended use. For example, a flowable composition is used when delivery via a syringe is desired; a putty is useful where the composition is to be placed in an exposed bone socket; and a solid may be used (alone in combination with a flowable or putty-like composition) when the final shape is known.

The curable admixture may be used in place of bone, such as in a tooth socket or other bony void (i.e., the spine), or may be placed in place of soft tissue, such as the area surrounding a tooth socket.

### Property of the Curable Admixture and the Cured Composite

Strength

It is preferred that the strength of the cured composite be from about 5 to 300 N/m²; more preferably from about 20 to 200 N/m²; and most desirably from about 50 to 200 N/m². The strength of the cured composite depends on a number of factors, such as the ratio between the bone substitute and crosslinkable prepolymer, and the crosslinking density of the cured composite.

In a preferred embodiment, that the cured composite has a compressive strength of at least 10 MPa. In one embodiment, the compressive strength is 20 to 30 MPa.

Porosity

High porosity is an important characteristic of the present invention. The bone substitute is porous to allow bone growth within the scaffold of the bone substitute, including the interstitial region between the particles when packed into an implant.

Hydrophobicity/Hydrophilicity

The hydrophobicity/hydrophilicity of the curable admixture and the cured composite should be carefully controlled. Preferably, the curable admixture and cured composite are sufficiently hydrophilic that cells adhere well to them. The hydrophobicity/hydrophilicity depends on a number of factors such as the hydrophobicity/hydrophilicity of the bone substitute and/or the crosslinkable prepolymer. For example, when the bone substitute is a PMMA/PHEMA based polymer particle, the ratio of PMMA (less hydrophilic) and PHEMA (more hydrophilic) affects the hydrophobicity/hydrophilicity. As another example, if the crosslinkable prepolymer is a polyanhydride instead of a polyethylene glycol, the curable admixture and the cured composite are more hydrophobic.

Viscosity

The viscosity of the curable admixture can vary widely. It depends on a number of factors such as the molecular weight of the ingredients in the curable admixture, and the temperature of the curable admixture. Typically, when the temperature is low, the curable admixture is more viscous; and, when the average molecular weight of the ingredients is high, it becomes more viscous. Different applications of the curable admixture also require different viscosities. For example, to be injectable, the admixture must be a free flowing liquid and, in other applications, it must be a moldable paste-like putty.

The viscosity of the curable admixture may be adjusted by formulating the crosslinkable prepolymer with a suitable amount of one or more biocompatible unsaturated functional monomers such as the ones described in U.S. Pat. Pub. 2003/114552 which are incorporated herein by reference.

Biodegradation/Bioresorption Duration

The time needed for biodegradation/bioresorption of the curable admixture and/or the cured composite can be varied widely, from days to years; preferably from weeks to months. The suitable biodegradation/bioresorption duration depends on a number of factors such as the speed of osteointegration, whether the compositions are functional and/or load-bearing, and/or the desirable rate of drug release. For example, osteointegration in an elderly woman is typically much slower than that in a 20 year old man. When osteointegration is slow, a composition having a long biodegradation/bioresorption time should be used. An immediately functional dental implant is load-bearing and must remain strong during osteointegration, so a long biodegradation/bioresorption composition is more suitable for application around such dental implant. If a therapeutic agent is intended to be released over a long period of time, a long biodegradation/bioresorption composition is more suitable.

Depending on the specific application, the time required can be manipulated based on a number of factors, e.g., the ratio of the bone substitute and the crosslinkable prepolymer. When the crosslinkable prepolymer contains more than one type of monomer, the ratio of the monomers also plays a crucial role in the degradation/resorption time. For example, when the crosslinkable prepolymer contains a mixture of dimethacrylated anhydrides of sebacic acid and 1,3-bis(p-carboxyphenxy)-propane, increasing the proportion of dimethacrylated anhydride of sebacic acid decreases the degradation/resorption time. Further, when the bone substitute is PMMA/PHEMA-based (known to be very slowly degradable), increasing the proportion of the bone substitute increases degradation time.

The degradation time is a function of the pH. For example, anhydrides are typically more susceptible to degradation in alkaline condition than in acidic condition.

The degradation time is a function of the hydrophobicity/hydrophilicity of the components. For example, when 1,3-bis(p-carboxyphenxy)-hexane (more hydrophobic) is replaced by 1,3-bis(p-carboxyphenxy)-propane (less hydrophobic), degradation time decreases.

The degradation time is also a function of geometrical shape, thickness, etc.

Where rapid degradation is sought, at least about 15% (w/w), preferably about 50% (w/w), of the cured composite degrades or resorbs in about 5-10 weeks, preferably in about 6-8 weeks.

On the other hand, for slow degradation at least about 15% (w/w), preferably about 50% (w/w), of the cured composite degrades or resorbs in about 6-12 months, preferably in about 9 months.

### Application of the Curable Admixture and the Cured Composite

Dental

The curable admixture and cured composite of the present invention can be used to fill extraction sockets; prevent or repair bone loss due to tooth extraction; repair jaw bone fractures; fill bone voids due to disease and trauma; stabilize an implant placed into an extraction socket and one placed into an edentulous jawbone to provide immediate function (e.g., chewing); provide ridge (of bone) augmentation; repair periodontal bone lesions; and provide esthetic gingiva reshaping and plumping. When the curable admixture and/or the cured composite is used for dental implant applications, preferably, the dental implant is partially or fully embedded into the cured composite according one of the following two methods:

Method (1):

Planting a dental implant into a bone and/or bone void;

at least partially embedding the dental implant by applying a curable admixture around the dental implant;

curing the curable admixture to form a cured composite; and

repeating steps (b) and (c) if necessary.

Method (2)
At least partially filling a bone void by applying the curable admixture;

curing the curable admixture to form a cured composite;

repeating steps (a) and (b) if necessary;

planting a dental implant into the bone by at least partially embedding the dental implant into the cured composite.

The curable admixture can be crosslinked by exposure to electromagnetic radiation and/or heat and applied using standard dental or surgical techniques. The curable admixture may be applied to the site where bone growth is desired and cured to form the cured composite and cured to form the cured composite. The curable admixture may also be pre-cast into a desired shape and size (e.g., rods, pins, screws, and plates) and cured to form the cured composite.

Orthopedic

The curable admixture and cured composite of the present invention can be used to repair bone fractures, fix vertebrae together, repair large bone loss (e.g., due to disease) and provide immediate function and support for load-bearing bones; to aid in esthetics (e.g., chin, cheek, etc.). The curable admixture can be applied using standard orthopedic or surgical techniques; e.g., it can be applied to a site where bone generation is desired and cured to form the cured composite. For example, the admixture can be applied into the intervertebral space. The curable admixture may also be pre-cast into a desired shape and size (e.g., rods, pins, screws, plates, and prosthetic devices such as for the spine, skull, chin and cheek) and cured to form the cured composite.

Drug Delivery

The curable admixture and cured composite of the present invention may be used to deliver therapeutic or diagnostic agents in vivo. Examples of drugs or agents which can be incorporated into such compositions include proteins, carbohydrates, nucleic acids, and inorganic and organic biologically active molecules. Specific examples include enzymes, antibiotics, antineoplastic agents, local anesthetics, hormones such as growth hormones, angiogenic agents, antiangiogenic agents, antibodies, neurotransmitters, psychoactive drugs, drugs affecting reproductive organs, and oligonucleotides such as antisense oligonucleotides.

### EXAMPLES

The following examples are intended to illustrate more specifically the embodiments of the invention. It will be understood that, while the invention as described therein is a specific embodiment, the description and the example are intended to illustrate and not limit the scope of the invention. Other aspects, advantages and modifications within the scope of the invention will be apparent to those skilled in the art to which the invention pertains.

### Example 1

This example illustrates the invention with the first embodiment of the crosslinkable prepolymer.

Curable admixtures are formed by mixing two crosslinkable prepolymers: (1) dimethacrylated anhydride of sebacic acid and (2) dimethacrylated anhydride of 1,3-bis(p-carboxyphenoxy)propane) with a bone substitute: (Bioplant® HTR®) as follows.

Formulation A

| Ingredient | Weight | |
|---|---|---|
| dimethacrylated anhydride of sebacic acid | 325 | mg |
| dimethacrylated anhydride of 1,3-bis(p- | 175 | mg |
| carboxyphenoxy) propane | | |
| DL-camphoquinone | 5 | mg |
| N-phenylglycine | 5 | mg |
| Bioplant ® HTR ® | 510 | mg |

The dimethacrylated anhydride of sebacic acid is formed by reacting sebacic acid with methacrylic anhydride by heating at reflux and the dimethacrylated anhydride of 1,3-bis(p-carboxyphenoxy)propane is formed by reacting 1,3-bis(p-carboxyphenoxy)propane with methacrylic anhydride by heating at reflux. DL-camphoquinone is used as a photoinitiator. This material is designed to be significantly resorbed in about 6-9 weeks when cured.

Formulation B

| Ingredient | Weight | |
|---|---|---|
| dimethacrylated anhydride of sebacic acid | 175 | mg |
| dimethacrylated anhydride of 1,3-bis(p- | 325 | mg |
| carboxyphenoxy) propane | | |
| DL-camphoquinone | 5 | mg |
| N-phenylglycine | 5 | mg |
| Bioplant ® HTR ® | 510 | mg |

This material is designed to be significantly resorbed in about 9 months.

### Example 2

This example illustrates the invention with the second embodiment of the crosslinkable prepolymer.

Formulation C

| Ingredient | Weight | |
|---|---|---|
| dimethacrylated anhydride of sebacic acid | 125 | mg |
| dimethacrylated anhydride of 1,3-bis(p- | 125 | mg |
| carboxyphenoxy) propane | | |
| Poly(1,3-bis(p-carboxyphenoxy) propane: | 250 | mg |
| sebacic acid) (80:20) | | |
| Irgacure 651 (Ciba-Geigy) | 1 | mg |
| Bioplant ® HTR ® | 501 | mg |

Poly(1,3-bis(p-carboxyphenoxy)propane:sebacic acid) (80:20) (“Poly(CPP:SA) (80:20)”) is a 80:20 (molar ratio) linear co-polymer of 1,3-bis(p-carboxyphenoxy)propane and sebacic acid. It is synthesized according to the procedure described in the Rosen et al. Biomaterials, 4, 131, (1983); Domb and Langer, J. Polym. Sci., 23, 3375, (1987).

### Example 3

This example illustrates the invention with the third embodiment of the crosslinkable prepolymer. The formulations are examples of a curable admixture formed by mixing (1) a crosslinkable prepolymer having at least two polymerizable terminal groups and a hydrophilic region with (2) bone substitute.

Formulation D

| Ingredient | Weight | |
|---|---|---|
| polyester bis-methacrylate | 254.6 | mg |
| demineralized bone powder | 256.2 | mg |
| DL-camphoquinone | 4.42 | mg |
| N-phenylglycine | 2.54 | mg |
| Bioplant ® HTR ® | 517.76 | mg |

The polyester bis-methacrylate is prepared according to the method described in Example 1 of WO01/74411.

Formulation E

| Ingredient | Weight | |
|---|---|---|
| poly(D,L-lactide₅₀-co-ε-caprolactone)- | 250 | mg |
| hexanediol_{20/1}-methacrylate | | |
| α-tricalcumphosphate | 250 | mg |
| DL-camphorquinone | 1.2 | mg |
| N-phenylglycine | 1.1 | mg |
| Bioplant ® HTR ® | 502.3 | mg |

The poly(D,L-lactide₅₀-co-ε-caprolactone)-hexanediol_{20/I}-methacrylate is prepared according to the method described in WO 01/74411.

### Example 4

The following experiment was conducted to study the bone ingrowth after extraction of molars and immediate fixation of an implant and placement of the curable admixture of the present invention. Formulation D of Example 3 was used.

Seven female sheep, ages 3 to 5 years, and thus having mature dentition, were used in the experiment. Two weeks prior to the extraction of teeth, the general health and dentition of the sheep were examined. If necessary, medication was used for de-vermification. Two days prior to the extraction, lateral and oblique pre-operation X-rays of the teeth to be removed were taken. One day prior to extraction, feeding was stopped and prophylactic AB (Excenel® RTU) and NSAID (Finadyne®) were administered. The next day (day 0) the P3 and P4 molars were extracted from both the left and right mandibles of the sheep. Preoperative medication of AB (Excenel® RTU) and Methylprednisolon (0.5 mg/kg, IM) was administered. The curable admixture in Example 3, Formulation D, was applied and cured in layers. The maximum thickness of each layer is about 5 mm. The light source was a standard dental 3M light in the visible light range. For each layer, the light was applied for 80 seconds.

In the left mandible, two titanium implants (Ankylos®), one normal and one modified with a square neck, were placed in one extraction socket. No implant was placed in the other socket. Bioplant® HTR® was mixed with Platelets Rich Plasma (PRP) and placed in the first socket around the implants as well as in the socket without implants. Bioplant® HTR® was then combined with the light curable polymer and placed in the first socket around the neck of the implants and in the occlusal part of the second socket without the implants. The strength of the mixture was from about 30 to about 40 N/m².

In the right mandible, two titanium implants (Ankylos®), one normal and one modified with a square neck, were placed in one extraction socket. No implant was placed in the other socket. Bioplant® HTR® was mixed with marrow bleeding and placed around the implants and in the socket without implants. Bioplant® HTR® was then combined with the light curable polymer and placed around the neck of the implants and in the occlusal part of the socket without the implants.

On days 1-3 AB (Excenel® RTU) (1 mg/kg) was administered. On day 30, 90 and 180 conventional and intra-oral X-rays were taken. On day 180, the sheep were euthanized and biopsies were performed for histological test.

### Example 5

The lower anterior incisor of Patient A was falling out due to advanced gingival and bone disease. Pre-operative X-ray revealed that there was almost no bone around the tooth (98% gone, bone resorbed because of gem infection). Abscess and infection were observed. The tooth was about 99% mobile and had to be held in place with fingers. If a normal apicoectomy were conducted, the tooth would not have survived (i.e., it would have fallen out).

After debridement of the area around the tooth, the curable admixture, Formulation D, was applied around the lower portion of the tooth in layers. Each layer was about 5 mm thick. After the application of each layer, the material in that layer was hardened in situ with blue dental light (source: 3M® Light) for about 80 seconds. The next layer was applied immediately after the previous layer was hardened. After the desirable stability and thickness was reached and esthetic shape or gingiva was obtained, the surgical flap was repositioned and sutured closed. The tooth was immediately stable, functional, and free of significant micromovement following the surgery. Twenty days and 3 months after surgery, the area was X-rayed to reveal significant bone growth.

### Example 6

The upper left central incisor of Patient B had a bone void of 98% due to the tooth extraction and the failed grafting of the socket area with Algipore® (General Medical, UK) graft material. Infection and graft failure resulted not only the loss of a portion of the Algipore® graft, but also the destruction of the entire buccal plate and the adjacent bone. The failed Algipore® was surrounded by infected soft tissue.

The failed Algipore® was first surgically removed. After debridement of the area, a large bone void was revealed. A metal implant was planted into the bone void with hand instrumentation and was stabilized by bone at the apex of the defect. There was only about 2 mm stabilization bone at the apex. Next, the curable admixture made according to Example 3, Formulation D, was applied around the implant in layers of approximately 5 mm or less and cured (hardened) with standard dental light for about 80 seconds. After the first layer was hardened, the next layer was added and cured. More layers were added and cured until the desired thickness for stability and esthetics was reached. Next, the soft tissue around the implant was sutured. An immediate post-operative temporary jacket was added and placed in function (e.g., contact for chewing). The implant was immediately functional, stable, and free of significant micromovement. X-rays taken 28 days after the surgery and implantation show bone growth was observed around the metal implant. There was no infection.

### Example 7

In addition to the synthesis method described in Example 1, methacrylated sebacic acids (MSA) and (1,3-bis(carboxyphenoxy))propyl dimethacrylate (CPPDM) were prepared according to the procedure described by Tarcha et al. J. Polym. Sci, Part A, Polym. Chem. (2001), 39, 4189. The MSA was synthesized by reacting sebacyl chloride and methacrylic acid at 0° C. in the presence of triethylamine and dichloromethane. The CPPDM was prepared by reacting methacrylocyl and 1,3-bis(p-caboxyphenoxy)propane (CPP) at 0° C. in the presence of triethylamine and dichloromethane.

### Example 8

### Samples Prepared

Nine samples were prepared as follows:

(1) 50 wt %:50 wt % LC:HTR (where LC is 100 wt % MSA);

(2) 45 wt %:45 wt %:10 wt % LC:HTR:sucrose (where LC is 100 wt % MSA);

(3) 50 wt %:50 wt % LC:HTR (where LC is 50 wt % MSA and 50 wt % CPPDM);

(4) 75 wt %:25 wt % LC:HTR (where LC is 100 wt % MSA);

(5) 75 wt %:25 wt % LC:HTR (where LC is 90 wt % CPPDM and 10 wt % MSA);

(6) 90 wt %:10 wt % LC:sucrose (where LC is 90 wt % CPPDM and 10 wt % MSA);

(7) 90 wt %:10 wt % LC:HTR (where LC is 90 wt % CPPDM and 10 wt % MSA);

(8) 90 wt %:5 wt %:5 wt % LC:HTR:sucrose (where LC is 90 wt % CPPDM, and 10 wt % MSA); and

(9) 100 wt % LC (where LC 90 wt % CPPDM and 10 wt % MSA).

HTR is abbreviation for Bioplant® HTR,® available from Bioplant Inc. (Norwalk, Conn.).

LC is abbreviation for light curable material. In these 9 samples, LC is MSA, CPPDM, or combination thereof.

MSA is abbreviation for methacrylated sebacic acid:

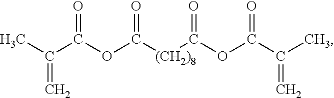

synthesized according to the procedure described by Tarcha et al. J. Polym. Sci, Part A, Polym. Chem. (2001), 39, 4189.

CPPDM is abbreviation for (1,3-bis(carboxyphenoxy))propyl dimethacrylate:

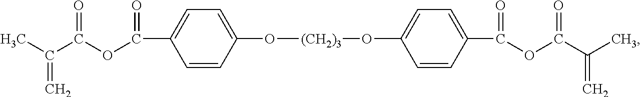

synthesized according to the procedure described by Tarcha et al. J. Polym. Sci, Part A, Polym. Chem. (2001), 39, 4189.

### Example 9

### Photopolyermization

To photopolymerize the samples in Example 8, an initiating system with ethyl 4-dimethylaminobenzoate in conjunction with an equal amount of camphorquinone was used. The ethyl 4-dimethylaminobenzoate and camphorquinone were dissolved in ethanol and added to each of the nine samples of Example 8 at 0.5 wt % relative to the total solids content (LC/HTR/sucrose combined).

The mixture was packed into teflon molds containing 5 mm holes, placed between two glass slides and exposed to a 450 nm visible light source to produce 1 mm thick disks for in vitro degradation experiments (Example 10 below) or 10 mm thick cylinders for in vitro mechanical strength testing (Example 11 below). Such in vitro tests provide good initial assessment as to whether the material would be useful for orthopedic or dental applications. For example, (1) high compressive yield strength indicates that the material is suitable for immediate dental implant purposes, because such dental implants would be able to withstand the biting and/or chewing forces immediately; and (2) percentage of mass loss within a certain time period indicates how fast the material would resorb in vivo and provide a situs for bone/tissue growth.

### Example 10

### Degradation Experiments

The disks prepared in Example 9 (5 mm in diameter×1 mm in thickness) were placed in individual tubes. The tubes were filled with approximately 1.5 ml of phosphate buffered saline (adjusted to pH 7.4) and the tubes were placed in a shaker incubator thermostatted at 37° C.; the buffer was removed and replaced every 1-2 days. Samples were removed periodically, weighed wet, then dried and reweighed. This allowed for calculation of the equilibrium swelling values as well as the mass loss over time. Data was collected in triplicate.

### Example 11

### Mechanical Strength Tests

The cylinders prepared in Example 9 (5 mm in diameter×10 mm in height) were used for the mechanical strength tests. Unconstrained uniaxial compression test were used to evaluate the mechanical properties of the cylinders at room temperature. Standard method was used to calibrate a 500 N load cell before testing. Five specimens of the each sample were mounted on a mechanical analyzer with the calibrated load cell. Specimens that broke at obvious flaws (e.g., water pocket or air pocket formation) were discarded. Strain was calculated from crosshead displacement. Stress was calculated from the load and cross-sectional area.

The ends of the samples were checked to make sure they are parallel to each other. Samples containing sucrose (i.e., Samples 2, 6, and 8) were soaked in de-ionized water overnight right before the testing date. All specimens were tested at 24° C. and ambient humidity.

The diameter of each sample was measured by a caliper to the nearest 0.01 mm at several points along its length. The minimum cross-sectional areas were calculated. The length of each specimen was measured to the nearest 0.01 mm. A concentric semi-circular mold was made to precisely mount the specimen at the center of the bottom anvil. Each specimen was mounted against the semi-circular mold between the surfaces of the anvils of the compression tool. The crosshead of the testing machine was adjusted until it just contacts the top of the compression tool plunger. The speed of the test was set at 1.3±0.3 mm/min. Loads and the corresponding compressive strain at appropriate intervals of strain were recorded to get the complete load-deformation curve. The maximum load carried by each specimen during the test (at the moment of rupture) was also recorded. If a specimen was relatively ductile, the speed was increased to 6 mm/min after the yield point had been reached; and the machine was run at this speed until the specimen breaks. The end point of the test was when the specimen was crushed to failure.

The following properties were calculated: (1) compressive yield strain: strain at the yield point; (2) compressive yield strength: stress at the yield point; and (3) crushing load: the maximum compressive force applied to the specimen, under the conditions of testing, that produces a designated degree of failure.

### Example 12

### Results and Discussion

The results of the degradation experiment (Example 10) and mechanical strength tests are summarized below.

TABLE 1
Results of testing for LC/BioPlant HTR formulations.

| | | | | Compressive | Compressive | Crushing | Integrity | | |
|---|---|---|---|---|---|---|---|---|---|
| | LC² | HTR | Sucrose | yield strain | yield strength | Load | lost | Swelling | % Mass loss |
| Sample¹ | (wt %) | (wt %) | (wt %) | (%) | (MPa) | (MPa) | (days) | wt % in water | (# days) |
| 1 | 50³ | 50 | 0 | — | 12.59 | — | 4 | slight amount, 50 wt % | 43 ± 2 (20) |
| | | | | | (±2.441) | | | | |
| 2 | 45³ | 45 | 10 | — | 4.365 | — | 4 | slight amount, 50 wt % | 49 ± 3 (18) |
| | | | | | (±1.334)⁶ | | | | |
| 3 | 50⁴ | 50 | 0 | — | — | — | 6 | slight amount, 50 wt % | 35 ± 2 (21) |
| 4 | 75³ | 25 | 0 | — | — | — | 8 | 100 wt % | 62 ± 4 (21) |
| 5 | 75⁵ | 25 | 0 | 6.285 | 18.81 | 19.06 | 11 | 50 wt % | 45 ± 2 (44) |
| | | | | (±1.30) | (±3.107) | (±3.15) | | | |
| 6 | 90⁵ | 0 | 10 | 5.186 | 9.295 | 22.44 | 11 | >200 wt % | 56 ± 6 (44) |
| | | | | (±0.4822) | (±1.249)⁶ | (±4.908) | | | |
| 7 | 90⁵ | 10 | 0 | 6.484 | 23.19 | — | 36 | slight amount, 50 wt % | 40 ± 4 (48) |
| | | | | (±0.3490) | (±1.612) | | | | |
| 8 | 90⁵ | 5 | 5 | 9.082 | 21.79 | 22.92 | 36 | slight amount, 50 wt % | 47 ± 2 (48) |
| | | | | (±1.229) | (±2.834)⁶ | (±2.584) | | | |
| 9 | 100⁵ | 0 | 0 | 5.878 | 11.67 | 14.36 | 56 | 75 wt % after 36 | 40 ± 3 (36) |
| | | | | (±0.8676) | (±3.028) | (±4.121) | | days | |

¹Photopolymerization conditions: 0.5 wt % camphorquinone, 0.5 wt % ethyl 4-dimethylaminobenzoate, λ = 450 nm
²MSA = methacrylated sebacic acid, CPPDM = (1,3-bis(carboxyphenoxy))propyl dimethacrylate
³composition = 100 wt % MSA
⁴composition = 50 wt % MSA/50 wt % CPPDM
⁵composition = 10 wt % MSA/90 wt % CPPDM
⁶soaked in deionized water to remove sucrose prior to testing

These results indicate that the materials of the present invention are suitable for various applications. For example, Samples (1)-(2) are suitable for very short term applications, delivery method for HTR to keep it in place temporarily; Sample (3) is suitable for short term applications and delivery method for HTR to keep it in place temporarily; Sample (4) is suitable for short term applications. The high swelling may lead to good integration and good cellular infiltration; Sample (5) is suitable for longer term applications where stability is needed for healing and integration because its mass loss is significantly slower than that of formulations with more MSA; Sample (6) is suitable for longer term applications where stability is needed for healing and integration because its swelling is significantly more than in any other formulation, which maybe useful for enhanced tissue integration; Sample (7) is suitable for a longer term formulation to promote bone growth while maintaining stability because it lacks swelling and degrades at a slower rate as compared to formulations with higher HTR contents; Sample (8) is suitable for longer term needs where the sucrose is added to allow for cellular infiltration, the presence of the sucrose may help improve tissue integration; and Sample (9) is suitable for systems where stability is vital to success.

### Example 13

### Multi-Stage Curing

A curable admixture is made according to Formulation F below.

Formulation F

| Ingredient | Weight | |
|---|---|---|
| dimethacrylated anhydride of sebacic acid | 300 | mg |
| dimethacrylated anhydride of 1,3-bis(p- | 300 | mg |
| carboxyphenoxy) propane | | |
| dimethacrylated polyethylene glycol | 400 | mg |
| α-tricalcium phosphate | 10 | mg |
| CaCO₃ | 10 | mg |
| CaCl₂ | 10 | mg |
| DL-camphoquinone | 5 | mg |
| N-phenylglycine | 5 | mg |
| Bioplant ® HTR ® | 1000 | mg |

The curable admixture made according to Formulation F is separated into equal portions: A and B. 5 mg of benzoyl peroxide (oxidizing component of a redox initiator system) is mixed into portion A. The resulting portion A is placed into one barrel of a multi-barrel syringe. 5 mg of N,N-dimethyl-p-toluidine (DMPT) (reducing component of a redox initiator system) is mixed into portion B. The resulting portion B is placed in to another barrel of the multi-barrel syringe.

Contents of the two barrels of the syringe are thoroughly mixed to partially cure the resulting mixture. The partially cured mixture is then applied to the tissue site and further cured by exposure to radiation. Barrel configurations can be either single with two-coaxial barrels or double, where one or both barrel(s) is covered to reduce light penetration.

### Example 14

This example illustrates the invention with the first embodiment of the crosslinkable prepolymer.

Curable admixtures are formed by mixing two crosslinkable prepolymers: (1) dimethacrylated anhydride of sebacic acid and (2) dimethacrylated anhydride of 1,3-bis(p-carboxyphenoxy)propane) with a bone substitute: (Bioplant® HTR®) as follows.

Formulation A

| Ingredient | Weight | |
|---|---|---|
| dimethacrylated anhydride of sebacic acid | 325 | mg |
| dimethacrylated anhydride of 1,3-bis(p- | 175 | mg |
| carboxyphenoxy) propane | | |
| DL-camphorquinone | 5 | mg |
| N-phenylglycine | 5 | mg |
| Bioplant ® HTR ® | 510 | mg |

The dimethacrylated anhydride of sebacic acid is formed by reacting sebacic acid with methacrylic anhydride by heating at reflux and the dimethacrylated anhydride of 1,3-bis(p-carboxyphenoxy)propane is formed by reacting 1,3-bis(p-carboxyphenoxy)propane with methacrylic anhydride by heating at reflux. DL-camphorquinone is used as a photoinitiator. This material is designed to be significantly resorbed in about 6-9 weeks when cured.

Formulation B

| Ingredient | Weight | |
|---|---|---|
| dimethacrylated anhydride of sebacic acid | 175 | mg |
| dimethacrylated anhydride of 1,3-bis(p- | 325 | mg |
| carboxyphenoxy) propane | | |
| DL-camphorquinone | 5 | mg |
| N-phenylglycine | 5 | mg |
| Bioplant ® HTR ® | 510 | mg |

This material is designed to be significantly resorbed in about 9 months.

### Example 15

This example illustrates the invention with the second embodiment of the crosslinkable prepolymer.

Formulaction C

| Ingredient | Weight | |
|---|---|---|
| dimethacrylated anhydride of sebacic acid | 125 | mg |
| dimethacrylated anhydride of 1,3-bis(p- | 125 | mg |
| carboxyphenoxy) propane | | |
| Poly(1,3-bis(p-carboxyphenoxy) propane: | 250 | mg |
| sebacic acid) (80:20) | | |
| Irgacure 651 (Ciba-Geigy) | 1 | mg |
| Bioplant ® HTR ® | 501 | mg |

Poly(1,3-bis(p-carboxyphenoxy)propane:sebacic acid) (80:20) (“Poly(CPP:SA) (80:20)”) is a 80:20 (molar ratio) linear co-polymer of 1,3-bis(p-carboxyphenoxy)propane and sebacic acid. It is synthesized according to the procedure described in the Rosen et al. Biomaterials, 4, 131, (1983); Domb and Langer, J. Polym. Sci., 23, 3375, (1987).

### Example 16

This example illustrates the invention with the third embodiment of the crosslinkable prepolymer. The formulations are examples of a curable admixture formed by mixing (1) a crosslinkable prepolymer having at least two polymerizable terminal groups and a hydrophilic region with (2) bone substitute.

Formulation D

| Ingredient | Weight | |
|---|---|---|
| polyester bis-methacrylate | 254.6 | mg |
| demineralized bone powder | 256.2 | mg |
| DL-camphorquinone | 4.42 | mg |
| N-phenylglycine | 2.54 | mg |
| Bioplant ® HTR ® | 517.76 | mg |

The polyester bis-methacrylate is prepared according to the method described in Example 1 of WO01/74411.

Formulation E

| Ingredient | Weight | |
|---|---|---|
| poly(D,L-lactide₅₀-co-ε-caprolactone)- | 250 | mg |
| hexanediol_{20/1}-methacrylate | | |
| α-tricalcumphosphate | 250 | mg |
| DL-camphorquinone | 1.2 | mg |
| N-phenylglycine | 1.1 | mg |
| Bioplant ® HTR ® | 502.3 | mg |

The poly(D,L-lactide₅₀-co-ε-caprolactone)-hexanediol_{20/I}-methacrylate is prepared according to the method described in WO 01/74411.

### Example 17

The following experiment was conducted to study the bone ingrowth after extraction of molars and immediate fixation of an implant and placement of the curable admixture of the present invention. Formulation D of Example 3 was used.

Seven female sheep, ages 3 to 5 years, and thus having mature dentition, were used in the experiment. Two weeks prior to the extraction of teeth, the general health and dentition of the sheep were examined. If necessary, medication was used for de-vermification. Two days prior to the extraction, lateral and oblique pre-operation X-rays of the teeth to be removed were taken. One day prior to extraction, feeding was stopped and prophylactic AB (Excenel® RTU) and NSAID (Finadyne®) were administered. The next day (day 0) the P3 and P4 molars were extracted from both the left and right mandibles of the sheep. Preoperative medication of AB (Excenel® RTU) and Methylprednisolon (0.5 mg/kg, IM) was administered. The curable admixture in Example 3, was applied and cured in layers. The maximum thickness of each layer is about 5 mm. The light source was a standard dental 3M light in the visible light range. For each layer, the light was applied for 80 seconds.

In the left mandible, two titanium implants (Ankylos®), one normal and one modified with a square neck, were placed in one extraction socket. No implant was placed in the other socket. Bioplant® HTR® was mixed with Platelets Rich Plasma (PRP) and placed in the first socket around the implants as well as in the socket without implants. Bioplant® HTR® was then combined with the light curable polymer and placed in the first socket around the neck of the implants and in the occlusal part of the second socket without the implants. The strength of the mixture was from about 30 to about 40 N/m².

In the right mandible, two titanium implants (Ankylos®), one normal and one modified with a square neck, were placed in one extraction socket. No implant was placed in the other socket. Bioplant® HTR® was mixed with marrow bleeding and placed around the implants and in the socket without implants. Bioplant® HTR® was then combined with the light curable polymer and placed around the neck of the implants and in the occlusal part of the socket without the implants.

On days 1-3 AB (Excenel® RTU) (1 mg/kg) was administered. On day 30, 90 and 180 conventional and intra-oral X-rays were taken. On day 180, the sheep were euthanized and biopsies were performed for histological test.

### Example 18

The lower anterior incisor of Patient A was falling out due to advanced gingival and bone disease. Pre-operative X-ray revealed that there was almost no bone around the tooth (98% gone, bone resorbed because of gem infection). Abscess and infection were observed. The tooth was about 99% mobile and had to be held in place with fingers. If a normal apicoectomy were conducted, the tooth would not have survived (i.e., it would have fallen out).

After debridement of the area around the tooth, the curable admixture, Formulation D, was applied around the lower portion of the tooth in layers. Each layer was about 5 mm thick. After the application of each layer, the material in that layer was hardened in situ with blue dental light (source: 3M® Light) for about 80 seconds. The next layer was applied immediately after the previous layer was hardened. After the desirable stability and thickness was reached and esthetic shape or gingiva was obtained, the surgical flap was repositioned and sutured closed. The tooth was immediately stable, functional, and free of significant micro-movement following the surgery.

### Example 19

The upper left central incisor of Patient B had a bone void of 98% due to the tooth extraction and the failed grafting of the socket area with Algipore® (General Medical, UK) graft material. Infection and graft failure resulted not only the loss of a portion of the Algipore® graft, but also the destruction of the entire buccal plate and the adjacent bone. The failed Algipore® was surrounded by infected soft tissue.

The failed Algipore® was first surgically removed. After debridement of the area, a large bone void was revealed. A metal implant was planted into the bone void with hand instrumentation and stabilized by bone at the apex of the defect. There was only about 2 mm stabilization bone at the apex. Next, the curable admixture made according to Example 3, Formulation D, was applied around the implant in layers of approximately 5 mm or less and cured (hardened) with standard dental light for about 80 seconds. After the first layer was hardened, the next layer was added and cured. More layers were added and cured until the desired thickness for stability and esthetics was reached. The complete graft with cured material of the present invention was shown to support the metal implant. Next, the soft tissue around the implant was sutured. An immediate post-operative temporary jacket was added and placed in function (e.g., contact for chewing). The implant was immediately functional, stable, and free of significant micro-movement. Bone growth was observed around the metal implant. There was no infection.

### Example 20

In addition to the synthesis method described in Example 1, methacrylated sebacic acids (MSA) and (1,3-bis(carboxyphenoxy))propyl dimethacrylate (CPPDM) were prepared according to the procedure described by Tarcha et al. J. Polym. Sci, Part A, Polym. Chem. (2001), 39, 4189. The MSA was synthesized by reacting sebacyl chloride and methacrylic acid at 0° C. in the presence of triethylamine and dichloromethane. The CPPDM was prepared by reacting methacrylocyl and 1,3-bis(p-caboxyphenoxy)propane (CPP) at 0° C. in the presence of triethylamine and dichloromethane.

### Example 21

Nine samples were prepared as follows:

50 wt %:50 wt % LC:Bioplant® HTR® (where LC is 100 wt % MSA);

45 wt %:45 wt %:10 wt % LC:Bioplant® HTR®:sucrose (where LC is 100 wt % MSA);

50 wt %:50 wt % LC:Bioplant® HTR® (where LC is 50 wt % MSA and 50 wt % CPPDM);

75 wt %:25 wt % LC:Bioplant® HTR® (where LC is 100 wt % MSA);

75 wt %:25 wt % LC:Bioplant® HTR® (where LC is 90 wt % CPPDM and 10 wt % MSA);

90 wt %:10 wt % LC:sucrose (where LC is 90 wt % CPPDM and 10 wt % MSA);

90 wt %:10 wt % LC:Bioplant® HTR® (where LC is 90 wt % CPPDM and 10 wt % MSA);

90 wt %:5 wt %:5 wt % LC:Bioplant® HTR® sucrose (where LC is 90 wt % CPPDM, and 10 wt % MSA); and

100 wt % LC (where LC 90 wt % CPPDM and 10 wt % MSA).

### Example 22

### Photopolymermization

To photopolymerize the samples in Example 8, an initiating system with ethyl 4-dimethylaminobenzoate in conjunction with an equal amount of camphorquinone was used. The ethyl 4-dimethylaminobenzoate and camphorquinone were dissolved in ethanol and added to each of the nine samples of Example 8 at 0.5 wt % relative to the total solids content (LC/HTR/sucrose combined).

The mixture was packed into Teflon molds containing 5 mm holes, placed between two glass slides and exposed to a 450 nm visible light source to produce 1 mm thick disks for in vitro degradation experiments (Example 10 below) or 10 mm thick cylinders for in vitro mechanical strength testing (Example 11 below). Such in vitro tests provide good initial assessment as to whether the material would be useful for orthopedic or dental applications. For example, (1) high compressive yield strength indicates that the material is suitable for immediate dental implant purposes, because such dental implants would be able to withstand the biting and/or chewing forces immediately; and (2) percentage of mass loss within a certain time period indicates how fast the material would resorb in vivo and provide a situs for bone/tissue growth.

### Example 23

### Degradation Experiments

In the disks prepared in Example 9 (5 mm in diameter×1 mm in thickness) were placed in individual tubes. The tubes were filled with approximately 1.5 ml of phosphate buffered saline (adjusted to pH 7.4) and the tubes were placed in a shaker incubator set to 37° C.; the buffer was removed and replaced every 1-2 days. Samples were removed periodically, weighed wet, then dried and reweighed. This allowed for calculation of the equilibrium swelling values as well as the mass loss over time. Data was collected in triplicate.

### Example 24

### Mechanical Strength Tests

The cylinders prepared in Example 9 (5 mm in diameter×10 mm in height) were used for the mechanical strength tests. Unconstrained uniaxial compression test were used to evaluate the mechanical properties of the cylinders at room temperature. Standard method was used to calibrate a 500 N load cell before testing. Five specimens of the each sample were mounted on a mechanical analyzer with the calibrated load cell. Specimens that broke at obvious flaws (e.g., water pocket or air pocket formation) were discarded. Strain was calculated from crosshead displacement. Stress was calculated from the load and cross-sectional area.

The ends of the samples were checked to make sure they are parallel to each other. Samples containing sucrose (i.e., Samples 2, 6, and 8) were soaked in de-ionized water overnight right before the testing date. All specimens were tested at 24° C. and ambient humidity.

The diameter of each sample was measured by a caliper to the nearest 0.01 mm at several points along its length. The minimum cross-sectional areas were calculated. The length of each specimen was measured to the nearest 0.01 mm. A concentric semi-circular mold was made to precisely mount the specimen at the center of the bottom anvil. Each specimen was mounted against the semi-circular mold between the surfaces of the anvils of the compression tool. The crosshead of the testing machine was adjusted until it just contacts the top of the compression tool plunger. The speed of the test was set at 1.3±0.3 mm/min. Loads and the corresponding compressive strain at appropriate intervals of strain were recorded to get the complete load-deformation curve. The maximum load carried by each specimen during the test (at the moment of rupture) was also recorded. If a specimen was relatively ductile, the speed was increased to 6 min/min after the yield point had been reached; and the machine was run at this speed until the specimen breaks. The end point of the test was when the specimen was crushed to failure.

The following properties were calculated: (1) compressive yield strain: strain at the yield point; (2) compressive yield strength: stress at the yield point; and (3) crushing load: the maximum compressive force applied to the specimen, under the conditions of testing, that produces a designated degree of failure.

### Example 25

### Results and Discussion

The results of the degradation experiment (Example 10) and mechanical strength tests are summarized below.

TABLE 1
Results of testing for LC/BioPlant ® HTR ® formulations.

| | | | | | | | | | % |
|---|---|---|---|---|---|---|---|---|---|
| | | | | | Compressive | | | | Mass |
| | LC² | HTR | | Compressive | yield | Crushing | Integrity | Swelling | loss |
| | (wt | (wt | Sucrose | yield strain | strength | Load | lost | wt % in | (# |
| Sample¹ | %) | %) | (wt %) | (%) | (MPa) | (MPa) | (days) | water | days) |
| 1 | 50³ | 50 | 0 | — | 12.59 | — | 4 | slight | 43 ± 2 |
| | | | | | (±2.441) | | | amount, | (20) |
| | | | | | | | | 50 wt % | |
| 2 | 45³ | 45 | 10 | — | 4.365 | — | 4 | slight | 49 ± 3 |
| | | | | | (±1.334)⁶ | | | amount, | (18) |
| | | | | | | | | 50 wt % | |
| 3 | 50⁴ | 50 | 0 | — | — | — | 6 | slight | 35 ± 2 |
| | | | | | | | | amount, | (21) |
| | | | | | | | | 50 wt % | |
| 4 | 75³ | 25 | 0 | — | — | — | 8 | 100 wt % | 62 ± 4 |
| | | | | | | | | | (21) |
| 5 | 75⁵ | 25 | 0 | 6.285 | 18.81 | 19.06 | 11 | 50 wt % | 45 ± 2 |
| | | | | (±1.30) | (±3.107) | (±3.15) | | | (44) |
| 6 | 90⁵ | 0 | 10 | 5.186 | 9.295 | 22.44 | 11 | >200 wt % | 56 ± 6 |
| | | | | (±0.4822) | (±1.249)⁶ | (±4.908) | | | (44) |
| 7 | 90⁵ | 10 | 0 | 6.484 | 23.19 | — | 36 | slight | 40 ± 4 |
| | | | | (±0.3490) | (±1.612) | | | amount, | (48) |
| | | | | | | | | 50 wt % | |
| 8 | 90⁵ | 5 | 5 | 9.082 | 21.79 | 22.92 | 36 | slight | 47 ± 2 |
| | | | | (±1.229) | (±2.834)⁶ | (±2.584) | | amount, | (48) |
| | | | | | | | | 50 wt % | |
| 9 | 100⁵ | 0 | 0 | 5.878 | 11.67 | 14.36 | 56 | 75 wt % | 40 ± 3 |
| | | | | (±0.8676) | (±3.028) | (±4.121) | | after 36 | (36) |
| | | | | | | | | days | |

¹Photopolymerization conditions: 0.5 wt % camphorquinone, 0.5 wt % ethyl 4-dimethylaminobenzoate, λ = 450 nm
²MSA = methacrylated sebacic acid, CPPDM = (1,3-bis(carboxyphenoxy))propyl dimethacrylate
³composition = 100 wt % MSA
⁴composition = 50 wt % MSA/50 wt % CPPDM
⁵composition = 10 wt % MSA/90 wt % CPPDM
⁶soaked in deionized water to remove sucrose prior to testing

These results indicate that the materials of the present invention are suitable for various applications. For example, Samples (1)-(2) are suitable for very short term applications, delivery method for Bioplant® HTR® to keep it in place temporarily; Sample (3) is suitable for short term applications and delivery method for Bioplant® HTR® to keep it in place temporarily; Sample (4) is suitable for short term applications. The high swelling may lead to good integration and good cellular infiltration; Sample (5) is suitable for longer term applications where stability is needed for healing and integration because its mass loss is significantly slower than that of formulations with more MSA; Sample (6) is suitable for longer term applications where stability is needed for healing and integration because its swelling is significantly more than in any other formulation, which maybe useful for enhanced tissue integration; Sample (7) is suitable for a longer term formulation to promote bone growth while maintaining stability because it lacks swelling and degrades at a slower rate as compared to formulations with higher Bioplant® HTR® contents; Sample (8) is suitable for longer term needs where the sucrose is added to allow for cellular infiltration, the presence of the sucrose may help improve tissue integration; and Sample (9) is suitable for systems where stability is vital to success.

### Example 26

### Multi-Stage Curing

In A curable admixture is made according to Formulation below.

| Ingredient | Weight | |
|---|---|---|
| dimethacrylated anhydride of sebacic acid | 300 | mg |
| dimethacrylated anhydride of 1,3-bis(p- | 300 | mg |
| carboxyphenoxy) propane | | |
| dimethacrylated polyethylene glycol | 400 | mg |
| α-tricalcium phosphate | 10 | mg |
| CaCO₃ | 10 | mg |
| CaCl₂ | 10 | mg |
| DL-camphorquinone | 5 | mg |
| N-phenylglycine | 5 | mg |
| Bioplant ® HTR ® | 1000 | mg |

The curable admixture made according to Formulation is separated into equal portions: A and B. 5 mg of benzoyl peroxide (oxidizing component of a redox initiator system) is mixed into portion A. The resulting portion A is placed into one barrel of a multi-barrel syringe. 5 mg of N,N-dimethyl-p-toluidine (DMPT) (reducing component of a redox initiator system) is mixed into portion B. The resulting portion B is placed in to another barrel of the multi-barrel syringe.

Contents of the two barrels of the syringe are thoroughly mixed to partially cure the resulting mixture. The partially cured mixture is then applied to the tissue site and further cured by exposure to radiation. Barrel configurations can be either single with two-coaxial barrels or double, where one or both barrel(s) is covered to reduce light penetration.

### Example 27

### Chemical and Light Initiator Components

Component A was prepared by mixing 0.5 g benzoyl peroxide and 0.5 g camphorquinone in 2 ml N-methyl-2-pyrrolidone (NMP). This mixture was stored in an opaque container in the refrigerator and was used for about a week before discarding.

A second version of component A was prepared by mixing 0.5 g benzoyl peroxide and 0.5 g camphorquinone in 10% v/v ethyl acetate. Then 2 ml poly(ethylene glycol) diacrylate, Mn ˜300 was added, and vortexed to mix. This mixture was stored in an opaque container in the refrigerator and was used for about a week before a fresh solution was made.

Component B was prepared by mixing 0.25 g 4-ethyl-dimethyl amino benzoate and 0.15 mL dimethyl para toluidine in 2 mL poly(ethylene glycol) diacrylate. Mn 258 (PEGDA˜300). This component was stored in the refrigerator and was used for about a week before discarding.

### Example 28

### Chemical Initiator Components

A solution of component A having only chemical curing properties was prepared by mixing 0.5 g benzoyl peroxide in 2 ml NMP. This mixture was stored in the refrigerator and was used for about a week before discarding.

A solution of component B having only chemical curing properties was prepared by mixing 1.0 mL dimethyl para toluidine in 2 mL poly(ethylene glycol) diacrylate. Mn 258 (PEGDA˜300). This component was stored in the refrigerator and was used for about a week before discarding.

For the following examples, the particular formulations used are:

| Ex- | | | | |
|---|---|---|---|---|
| ample | Formulation | | | |
| 29 | 90% MCPP | 10% PEG DMA | | |
| 30 | 90% MCPP | 10% PEG DMA | | formulated |
| | | | | with 25% |
| | | | | filler |
| 31 | 75% MCPP | 25% PEG DMA | | |
| 32 | 75% MCPP | 25% PEG DMA | | formulated |
| | | | | with 25% |
| | | | | filler. |
| 33 | 90% MCPP | 10% PEG DMA | 5% SA | |
| 34 | 50% MCPP | 25% PEGDMA600 | 25% MSA | |
| 35 | 40% MCPP | 15% PEG DMA | 15% MSA | 30% CaCO₃ |
| 36 | 50% MCPP | 25% PEGDMA600 | 25% MSA | formulated |
| | | | | with 25% |
| | | | | Bioplant ® |
| | | | | HTR ® |
| 37 | 50% MCPP | 25% PEGDMA600 | 25% MSA | formulated |
| | | | | with 50% |
| | | | | Bioplant ® |
| | | | | HTR ® |
| 38 | 65% MCPP | 15% PEGDMA600 | 10% MSA | 10% CaCO₃ |
| 39 | 65% MCPP | 15% PEGDMA600 | 20% MSA | |
| 40 | 65% MCPP | 15% PEGDMA600 | 20% MSA | formulated |
| | | | | with 30% |
| | | | | Bioplant ® |
| | | | | HTR ® |
| 41 | 90% MCPP | 10% PEGDMA600- | | |
| | | chemical cure | | |
| 42 | 75% MCPP | 25% PEGDMA600- | | |
| | | chemical cure | | |
| 43 | 75% MCPP | 25% PEGDMA600- | | formulated |
| | | chemical cure | | with 25% |
| | | | | Bioplant ® |
| | | | | HTR ® |
| 44 | 70% MCPP | 25% PEGDMA600 | 5% MSA | |
| 45 | 70% MCPP | 25% PEGDMA600 | 5% MSA- | |
| | | | chemical | |
| | | | cure | |
| 46 | 55% MCPP | 20% PEGDMA600 | 15% MSA | 10% CaCO₃ |
| 47 | 55% MCPP | 20% PEGDMA600 | 15% MSA - | 10% CaCO₃ |
| | | | chemical | |
| | | | cure | |

### Example 29

MCPP was combined with the PEG DMA and mixed thoroughly (for 2-5 minutes). Component A from Example 27 was added and mixed until the color and consistency was evenly dispersed. Then component B from Example 27 was added and mixed thoroughly. Because of the high viscosity of the sample, care must be taking during mixing of both component A and component B to obtain a homogeneous mixture. The mixture was allowed to stand for approximately 30 seconds with occasional mixing before transfer to a mold 12 mm in diameter where it was packed down to remove air pockets. Dental blue light was directed onto the sample for 1 minute (or up to 2 minutes for other preferred applications), during which the sample was rotated to promote uniformity. After cooling, the sample was removed from the mold.

90% MCPP, 10% PEG DMA

| Ingredient | Weight | |
|---|---|---|
| Methacrylated poly(1,3-bis(p-carboxy- | 4.5 | g |
| phenoxy) propane | | |
| dimethacrylated polyethylene glycol 600 | 500 | μl |
| Component A | 100 | μl |
| Component B | 100 | μl |

For testing of this sample, the sample was removed from the mold and cut down to a 25.4 mm height and placed in phosphate buffered saline (PBS) at 37° C. for 24 hours. Compressive strength testing demonstrates a max load of 870±326 N and a max stress of 8±3 mPa.

### Example 30

The sample can be prepared as described in Example 29, using the Component A and Component B as prepared in Example 27. The Bioplant® HTR® will be stirred into the sample with Component A.

90% MCPP, 10% PEG DMA—Formulated with 25% Filler

| Ingredient | Weight | |
|---|---|---|
| dimethacrylated anhydride of 1,3-bis(p- | 3.375 | g |
| carboxyphenoxy) propane | | |
| dimethacrylated polyethylene glycol 600 | 375 | μl |
| Bioplant ® HTR ® | 1.25 | g |
| Component A | 100 | μl |
| Component B | 100 | μl |

The Bioplant® HTR® in this formulation adds strength and increase resorption time.

### Example 31

The sample of was prepared as described in Example 29, using the Component A and Component B as prepared in Example 27.

75% MCPP, 25% PEG DMA

| Ingredient | Weight | |
|---|---|---|
| dimethacrylated anhydride of 1,3-bis(p- | 3.75 | mg |
| carboxyphenoxy) propane | | |
| dimethacrylated polyethylene glycol 600 | 1.25 | ml |
| Component A | 100 | μl |
| Component B | 100 | μl |

After 24 hours preconditioning in PBS at 37° C., compressive strength was 7 MPa at 748 N max load for 1 sample and 10 MPa at 1174 N max load for another.

### Example 32

The sample of was prepared as described in Example 29, using the Component A and Component B as prepared in Example 27. The Bioplant® HTR® was stirred into the sample with Component A.

75% MCPP, 25% PEG DMA—Formulated with 25% Filler

| Ingredient | Weight | |
|---|---|---|
| dimethacrylated anhydride of 1,3-bis(p- | 2.813 | g |
| carboxyphenoxy) propane | | |
| dimethacrylated polyethylene glycol 600 | 0.932 | ml |
| Bioplant ® HTR ® | 1.25 | g |
| Component A | 100 | μl |
| Component B | 100 | μl |

After 24 hours preconditioning in PBS at 37° C., compressive strength was 11 MPa at 1201 N max load in one sample and 14 MPa at 1645 N max load in another.

### Example 33

MCPP was combined with the PEG DMA and mixed thoroughly (for 2-5 minutes). Then the SA was mixed with the MCPP/PEG mixture. Component A from Example 27 was added and mixed until the color and consistency was evenly dispersed. Then component B from Example 27 was added and mixed thoroughly. The mixture was allowed to stand for approximately 30 seconds with occasional mixing before transfer to a mold 12 mm in diameter where it was packed down to remove air pockets. Dental blue light was directed onto the sample for 1 minute, during which the sample was rotated to promote uniformity. After cooling, the sample was removed from the mold.

90% MCPP, 10% PEG DMA—with 5% SA

| Ingredient | Weight | |
|---|---|---|
| dimethacrylated anhydride of 1,3-bis(p- | 855 | mg |
| carboxyphenoxy) propane | | |
| dimethacrylated polyethylene glycol 600 | 95 | mg |
| dimethacrylated anhydride of sebacic acid | 50 | mg |
| Component A | 20 | μl |
| Component B | 20 | μl |

This formulation is made having increased plasticity and easier production than the admixture without SA.

### Example 34

The sample can be prepared as described in Example 33, using the Component A and Component B as prepared in Example 27 and where MSA is used.

50% MCPP, 25% MSA, 25% PEGDMA600

| Ingredient | Weight | |
|---|---|---|
| dimethacrylated anhydride of 1,3-bis(p- | 500 | mg |
| carboxyphenoxy) propane | | |
| dimethacrylated polyethylene glycol | 250 | mg |
| dimethacrylated anhydride of sebacic acid | 250 | mg |
| Component A | 20 | μl |
| Component B | 20 | μl |

This sample is designed for fast resorption properties.

### Example 35

The sample can be prepared as described in Example 33, using the Component A and Component B as prepared in Example 27. The CaCO₃ is stirred in with component A, MCPP, and PEG-DM.

40% MCPP, 15% PEG DMA, 15% MSA, 30% CaCO₃ Filler

| Ingredient | Weight | |
|---|---|---|
| dimethacrylated anhydride of 1,3-bis(p- | 2 | g |
| carboxyphenoxy) propane | | |
| dimethacrylated polyethylene glycol | 0.75 | g |
| dimethacrylated anhydride of sebacic acid | 0.75 | g |
| CaCO₃ | 1.5 | g |
| Component A | 100 | μl |
| Component B | 100 | μl |

This sample is designed for fast resorption properties, lower viscosity, moderate strength

### Example 36

The sample can be prepared as described in Example 33, using the Component A and Component B as prepared in Example 27.

50% MCPP, 25% MSA, 25% PEGDMA600—Formulated with 25% Bioplant® HTR® Filler

| Ingredient | Weight | |
|---|---|---|
| dimethacrylated anhydride of 1,3-bis(p- | 1.875 | g |
| carboxyphenoxy) propane | | |
| dimethacrylated polyethylene glycol | 0.938 | g |
| dimethacrylated anhydride of sebacic acid | 0.938 | g |
| Bioplant ® HTR ® | 1.25 | g |
| Component A | 100 | μl |
| Component B | 100 | μl |

This sample provides the strength and slow rate of degradation due to the HTR filler component as well as the high strength from the addition of the MSA.

### Example 37

The sample can be prepared as described in Example 33, using the Component A and Component B as prepared in Example 27.

50% MCPP, 25% MSA, 25% PEGDMA600—Formulated with 50% Bioplant® HTR® Filler.

| Ingredient | Weight | |
|---|---|---|
| dimethacrylated anhydride of 1,3-bis(p- | 1.25 | g |
| carboxyphenoxy) propane | | |
| dimethacrylated polyethylene glycol | 0.625 | g |
| dimethacrylated anhydride of sebacic acid | 0.625 | g |
| Bioplant ® HTR ® | 2.5 | g |
| Component A | 100 | μl |
| Component B | 100 | μl |

This sample provides the strength and slow rate of degradation due to the HTR filler component as well as the high strength from the addition of the MSA. A similar formulation can be made with 25% or 30% Bioplant® HTR®.

### Example 38

The sample can be prepared as described in Example 33, using the Component A and Component B as prepared in Example 27.

65% MCPP, 10% MSA, 15% PEGDMA600, 10% CaCO₃

| Ingredient | Weight | |
|---|---|---|
| dimethacrylated anhydride of 1,3-bis(p- | 3.25 | g |
| carboxyphenoxy) propane | | |
| dimethacrylated polyethylene glycol | 0.75 | g |
| dimethacrylated anhydride of sebacic acid | 0.50 | g |
| CaCO₃ | 0.50 | g |
| Component A | 100 | μl |
| Component B | 100 | μl |

This sample is designed for strength and biodegradation times shorter than can be obtained with the addition of Bioplant® HTR®.

### Example 39

The sample can be prepared as described in Example 33, using the Component A and Component B as prepared in Example 27.

65% MCPP, 15% MSA, 20% PEGDMA600

| Ingredient | Weight | |
|---|---|---|
| dimethacrylated anhydride of 1,3-bis(p- | 3.25 | g |
| carboxyphenoxy) propane | | |
| dimethacrylated polyethylene glycol | 0.75 | g |
| dimethacrylated anhydride of sebacic acid | 1.0 | g |
| Component A | 100 | μl |
| Component B | 100 | μl |

This sample is formulated for high strength.

### Example 40

The sample can be prepared as described in Example 33, using the Component A and Component B as prepared in Example 27 with the addition of Bioplant® HTR®.

65% MCPP, 15% MSA, 20% PEGDMA600—Formulated with 30% Bioplant® HTR®

| Ingredient | Weight | |
|---|---|---|
| dimethacrylated anhydride of 1,3-bis(p- | 3.25 | g |
| carboxyphenoxy) propane | | |
| dimethacrylated polyethylene glycol | 0.75 | g |
| dimethacrylated anhydride of sebacic acid | 1.0 | g |
| Bioplant ® HTR ® | 1.5 | g |
| Component A | 100 | μl |
| Component B | 100 | μl |

This sample is formulated for high strength and good bone growth characteristics.

### Example 41

MCPP was combined with the PEG and mixed thoroughly (for 2-5 minutes) until the texture was uniform. The mixture was then transferred to a mold 12 mm in diameter where it was loosely packed. Component A from Example 28 was added and mixed thoroughly (2-3 minutes). Then component B from Example 28 was added and mixed thoroughly (2-3 minutes). The material was packed down in the mold to compress and remove air pockets (15-30 sec.) The sample was left in the mold for 2-3 hours for curing.

90% MCPP, 10% MPEG—Chemical Cure

| Ingredient | Weight | |
|---|---|---|
| Methacrylated poly(1,3-bis(p- | 4.5 | g |
| carboxyphenoxy) propane | | |
| PEG DMA 600 | 500 | μl |
| Component A | 100 | μl |
| Component B | 100 | μl |

For testing of this sample, the sample was removed from the mold and cut down to 25.4 mm height and placed in phosphate buffered saline (PBS) at 37° C. for 24 hours. Compressive strength for two samples using this formulation were: (a) 4 mPa at 489 N, and (b) 15 MPa at 1675 N.

### Example 42

The sample of was prepared as described in Example 28, using the Component A and Component B as prepared in Example 28.

75% MCPP, 25% MPEG—Chemical Cure

| Ingredient | Weight | |
|---|---|---|
| Methacrylated poly(1,3-bis(p- | 3.75 | g |
| carboxyphenoxy) propane | | |
| PEG DMA 600 | 1.25 | ml |
| Component A | 100 | μl |
| Component B | 100 | μl |

After 24 hours preconditioning in PBS at 37° C., compressive strength was 12 MPa at 1324 N.

### Example 43

The sample was prepared as described in Example 28, using the Component A and Component B as prepared in Example 28.

75% MCPP, 25% MPEG, formulated with 25% Bioplant® HTR® Filler—Chemical Cure

| Ingredient | Weight | |
|---|---|---|
| Methacrylated poly(1,3-bis(p- | 2.81 | g |
| carboxyphenoxy) propane | | |
| PEG DMA 600 | 938 | μl |
| Bioplant ® HTR ® | 1.25 | g |
| Component A | 100 | μl |
| Component B | 100 | μl |

After 24 hours preconditioning in PBS at 37° C., compressive strength was 8 MPa at 938 N.

### Example 44

MCPP was combined with the PEG DMA and mixed thoroughly. Then the MSA was mixed with the MCPP/PEG mixture for approximately 10 minutes. Component A from Example 27 was added and mixed for about 4 minutes. Then component B from Example 27 was added and mixed thoroughly (about 1 minute). The mixture was poured into a mold having a 6.3 mm inner diameter and a length of 12.6 mm. Dental blue light was directed onto the sample for 1 minute, with the sample rotated after 30 seconds. The sample was allows to cure for 2-3 hours and the mold was removed. The sample was then filed down to the desired length for compression testing. The samples were left in phosphate buffered saline solution at 37° C. for 24 hours before testing for strength.

70% MCPP, 25% PEGDMA600, 5% MSA

| Ingredient | Weight | |
|---|---|---|
| Methacrylated poly(1,3-bis(p- | 0.7 | g |
| carboxyphenoxy) propane | | |
| PEG DMA 600 | 250 | μl |
| MSA | 50 | μl |
| Component A | 20 | μl |
| Component B | 20 | μl |

### Example 45

The sample was prepared as described in Example 31, except that light was not used on this sample. Component A and Component B were used as prepared in Example 28.

70% MCPP 25% PEGDMA600 5% MSA—Chemical Cure

| Ingredient | Weight | |
|---|---|---|
| Methacrylated poly(1,3-bis(p- | 0.7 | g |
| carboxyphenoxy) propane | | |
| PEG DMA 600 | 250 | μl |
| Bioplant ® HTR ® | 50 | μl |
| Component A | 20 | μl |
| Component B | 20 | μl |

### Example 46

The sample was prepared as described in Example 31, using the Component A and Component B as prepared in Example 27. The CaCO₃ was added after the MSA was mixed with the MCPP and PEG DMA and mixed for 10 minutes.

55% MCPP 20% PEGDMA600 15% MSA 10% CaCO₃

| Ingredient | Weight | |
|---|---|---|
| Methacrylated poly(1,3-bis(p- | 0.55 | g |
| carboxyphenoxy) propane | | |
| PEG DMA 600 | 200 | μl |
| CaCO₃ | 100 | μg |
| Component A | 20 | μl |
| Component B | 20 | μl |

### Example 47

The sample was prepared as described in Example 33 except that no light was used. Component A and Component B were used as prepared in Example 28.

55% MCPP 20% PEGDMA600 15% MSA—Chemical Cure 10% CaCO₃

| Ingredient | Weight | |
|---|---|---|
| Methacrylated poly(1,3-bis(p- | 0.55 | g |
| carboxyphenoxy) propane | | |
| PEG DMA 600 | 200 | μl |
| CaCO₃ | 100 | μg |
| Component A | 20 | μl |
| Component B | 20 | μl |

### Example 48

Four different formulations were prepared as described above with the addition of camphorquinone and ethyl 4-dimethylaminobenzoate. The samples were placed in tibia and zygoma defects in rabbits. These formulations provide different lengths of time for resorption, i.e., short acting and longer acting. The 4 formulations tested in rabbits are:

| | | | | |
|---|---|---|---|---|
| F1 | 90% | MCPP | 10% MSA | |
| F2 | 90% | MCPP | 10% MSA | formulated with 10% |
| | | | | Bioplant ® HTR ® and CaCO₃ |
| F3 | 90% | MCPP | 10% MSA | formulated with 25% |
| | | | | Bioplant ® HTR ® and CaCO₃ |
| F4 | 100% | MSA | | formulated with 25% |
| | | | | Bioplant ® HTR ® |

10% sucrose and 10% gellaten were added as porogens.

The polymer samples were placed into defects in the rabbit tibia and zygoma (6 mm trephine on each), hardened with light, and evaluated at 4 or 8 weeks. Histological results show polymer resorption and bone growth at 4 and 8 weeks. Voids present in locations where the polymer materials were initially placed indicate the resorption of the polymer with subsequent regrowth of bone into the void. Generally, the anhydride polymer material resorbed and new bone formed and bridged normally. The materials used in this study did not appear to cause significant inflammation, rejection, necrosis, or foreign body reaction. Controls included empty (non-grafted) control defects. There were no adverse events with the anhydride alone, a anhydride and Bioplant® HTR® or control sites in any location in any animal.

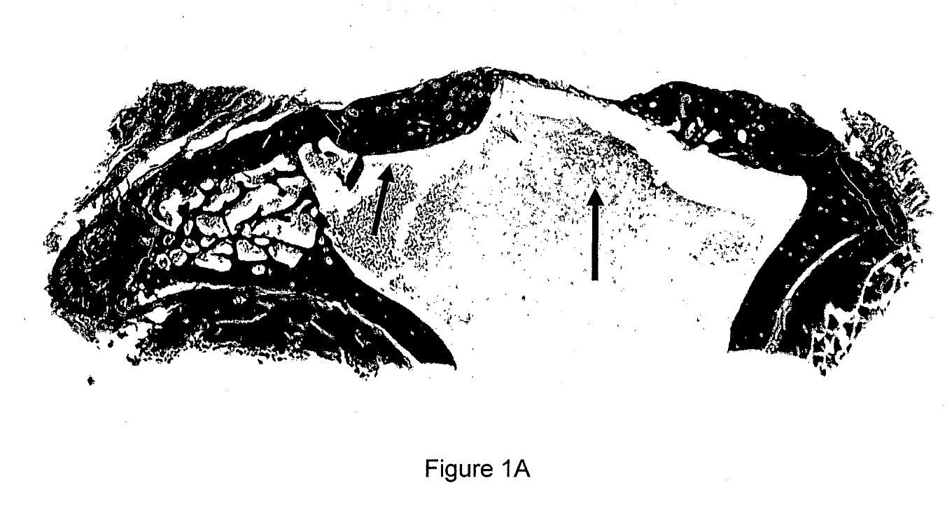

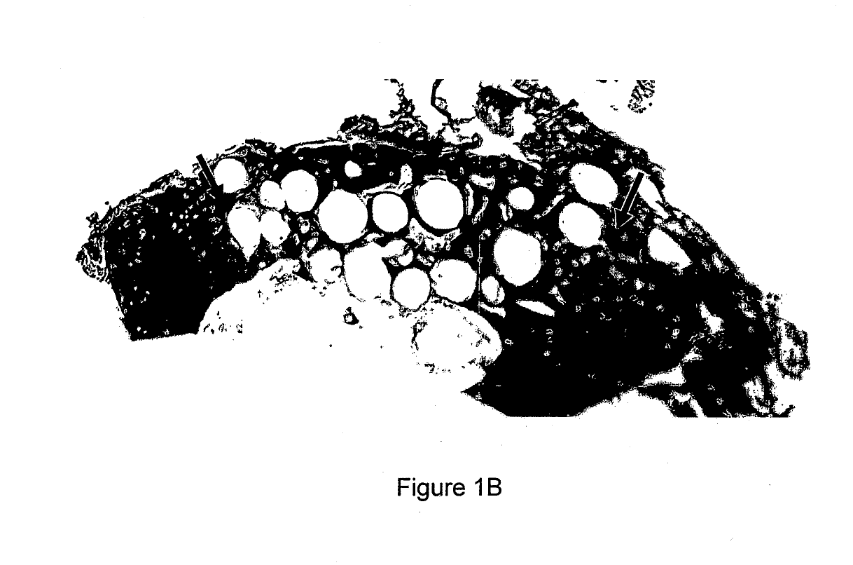

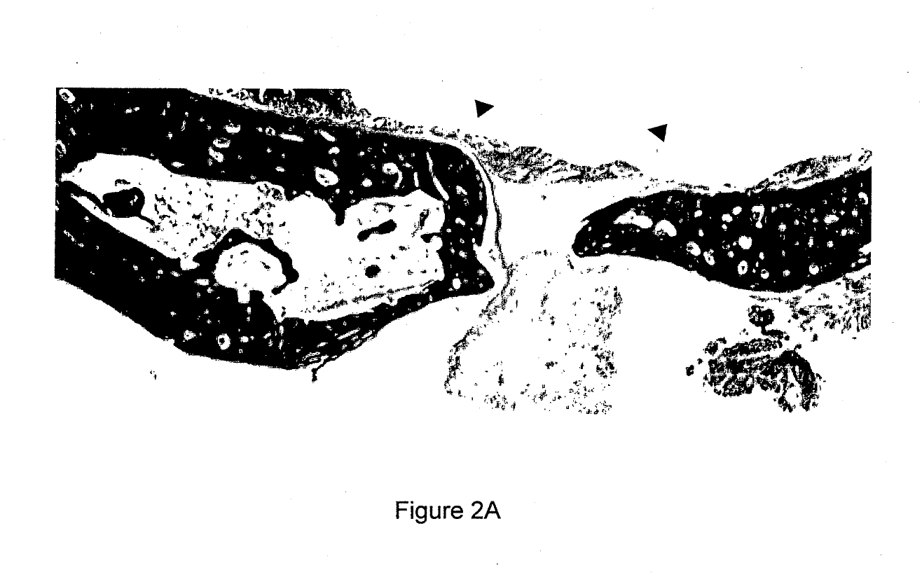

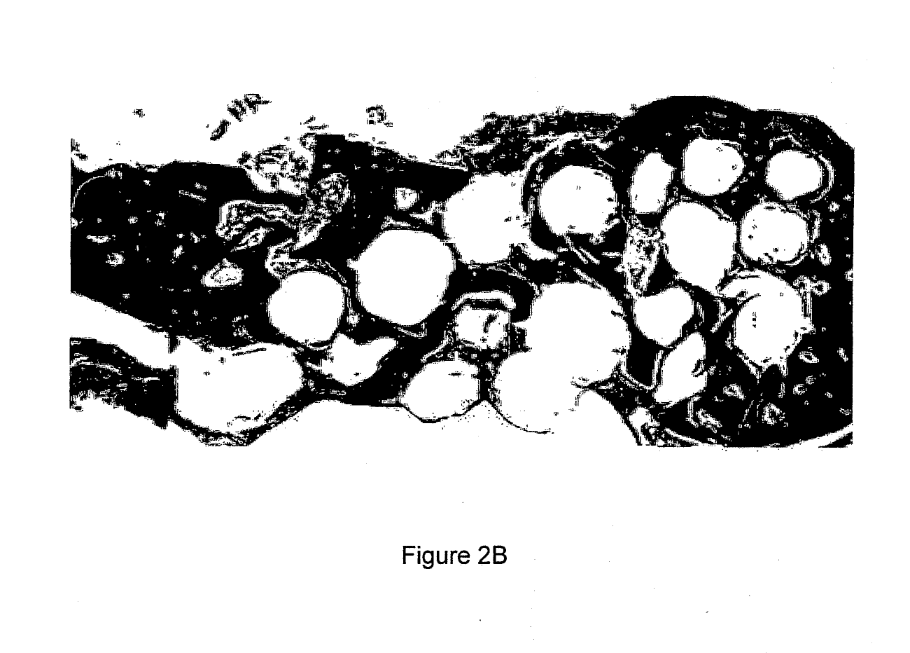

Generally, new bone was seen to bridge most of the defect in either the tibia (FIGS. 1A and 1B) or zygoma (FIGS. 2A and 2B) samples by 8 weeks in both the control (FIGS. 1A and 2A) and light hardened polymer containing the bone substitute Bioplant® HTR® (FIGS. 1B and 2B). Fingers of new bone growth are seen near the periphery of the bony defect for both. The presence of the anhydride polymer and Bioplant® HTR® maintained and helped reconstitute the dimensions of the defects and provided scaffolding for the bone growth, as new growth was observed at the periphery where the anhydride was observed and resorbing as well around the Bioplant® HTR® materials in the depth of the defect.

1. A crosslinkable bone substitute material comprising a plurality of micron sized particles and crosslinkable pre-polymer,
wherein each particle comprises a core layer comprising a first polymeric material and a coating comprising a second polymeric material which is different from the first polymeric material; and wherein the micron sized particles are coated at least in part with a crosslinkable pre-polymer capable of crosslinking to form a polymer network.

2. The crosslinkable bone substitute material of claim 1, wherein the first polymeric material comprises an acrylic polymer.

3. The crosslinkable bone substitute of claim 1, wherein the first polymeric material is poly(methyl methacrylate) (PMMA).

4. The crosslinkable bone substitute of claim 1, wherein the second polymeric material is polymeric hydroxyethyl methacrylate (PHEMA).

5. The crosslinkable bone substitute of claim 1, wherein the micron sized particles further comprise calcium hydroxide, calcium carbonate, or a copolymer thereof.

6. The crosslinkable bone substitute of claim 1, wherein the micron sized particles further comprise a non-binding agent.

7. The crosslinkable bone substitute of claim 6, wherein the non-binding agent is barium sulfate.

8. The crosslinkable bone substitute of claim 1, wherein the crosslinkable pre-polymer comprises a monomer and/or oligomer having a biodegradable ester linkage.

9. The crosslinkable bone substitute of claim 1, wherein the crosslinkable prepolymer comprises (meth)acrylate.

10. A crosslinked bone substitute material comprising a plurality of micron sized particles wherein each particle comprises a core layer comprising a first polymeric material and a coating comprising a second polymeric material which is different from the first polymeric material; and wherein the micron sized particles are crosslinked electrostatically or chemically to each other by a crosslinked moiety coating at least a portion of each particle.

11. The crosslinked bone substitute material of claim 10, wherein the first polymeric material comprises an acrylic polymer.

12. The crosslinked bone substitute of claim 10, wherein the first polymeric material is poly(methyl methacrylate) (PMMA).

13. The crosslinked bone substitute of claim 10, wherein the second polymeric material is polymeric hydroxyethyl methacrylate (PHEMA).

14. The crosslinked bone substitute of claim 10, wherein the micron sized particles further comprise calcium hydroxide, calcium carbonate, or a copolymer thereof.

15. The crosslinked bone substitute of claim 10, wherein the micron sized particles further comprise a non-binding agent.

16. The crosslinked bone substitute of claim 15, wherein the non-binding agent is barium sulfate.

17. The crosslinked bone substitute of claim 10, wherein the crosslinkable pre-polymer comprises a monomer and/or oligomer having a biodegradable ester linkage.

18. The crosslinked bone substitute of claim 10, wherein the crosslinkable prepolymer comprises (meth)acrylate.

19. The crosslinked bone substitute of claim 10, wherein the bone substitute is crosslinked using a photoinitiator and the application of light to the bone substitute.

20. A method of promoting bone generation comprising the steps of applying the crosslinkable bone substitute of claim 1 to an area in need of bone generation and crosslinking the bone substitute; wherein the one substitute promotes and/or induces bone generation and wherein the bone substitute contains pores into which bone tissue can grow.
